SEQUENCE LISTING (14 sequences)

SEQ ID NO: 1 - DNA, 366 bp, Mus musculus
gatgtgcagcttcaggagtcgggacctggcctggtgaaaccttctcagtctctgtccctcacctgcactgtcactggctactcaatcaccagtgattatgcctggaactggatccggcagtttccaggaaacaagctggagtggatgggctacataagctacagtggtagcactagctaccacccatctctcaaaagtcgaatctctatcactcgagacacatccaagaaccagttcttcctgcagttgaattctgtgactactgaggacacagccacatattactgtacaactatcattaactatgataagtacgactggtacttcgatgtctggggcgcagggaccacggtcaccgtctcctca

SEQ ID NO: 2 - protein, 122 aa, Mus musculus
DVQLQESGPGLVKPSQSLSLTCTVTGYSITSDYAWNWIRQFPGNKLEWMGYISYSGSTSYHPSLKSRISITRDTSKNQFFLQLNSVTTEDTATYYCTTIINYDKYDWYFDVWGAGTTVTVSS

SEQ ID NO: 3 - DNA, 332 bp, Mus musculus
  misc_feature (32)..(34): n is a, c, g, or t
  misc_feature (67)..(69): n is a, c, g, or t
tacattgtgctgacacagtctcctgcttcctnnnttgtgtctctgaggcagagggccaccatctcannngggccagccaaagtgtcaggacatcaagttatagttatatacactggtaccaacagaaaccaggacagccacccaaactcctcatcaagtatgcatccagcctagaatctggggtccctgccaggttcagtggcagtgggtctgggacagacttcaccctcaacatccatcctgtggaggaggaggatactgcaacatattactgtcagcacagttgggagattccgtggacgttcggtggaggcaccacactggaaatcaga

SEQ ID NO: 4 - protein, 111 aa, Mus musculus
DIVLTQSPASLVVSLRQRATISCRASQSVRTSSYSYIHWYQQKPGQPPKLLIKYASSLESGVPARFSGSGSGTDFTLNIHPVEEEDTATYYCQHSWEIPWTFGGGTTLEIR

SEQ ID NO: 5 - DNA, 827 bp, Mus musculus
  misc_feature (36)..(36): n is a, c, g, or t
  misc_feature (69)..(72): n is a, c, g, or t
  misc_feature (664)..(666): n is a, c, g, or t
  misc_feature (700)..(702): n is a, c, g, or t
aagcttatgaatatgcaaatcctctgaatctacatngtaaatataggtttgtctataccacaaacagannnncatgagatcacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactccgatgtgcagcttcaggagtcgggacctggcctggtgaaaccttctcagactctgtccctcacctgcactgtctctggctactcaatgaccagtgattatgcctggaactggattcggcagtttccaggaaaggggctggagtggatcggctacataagctacagtggtagcactacctaccacccatctgtcaaaagtcgaatcactatctctcgagacacatccaagaaccagttcttcctgcagatgaactctgtgactactgaggacacagccacatattactgtacaactatcattaactatgataagtacgactggtacttcgattactggggccaagggaccacggtcaccgtctcctcaggtggtccttacaacctctctcttctattcagcttaannngattttactgcatttgttgggggggaaatgtgtnnntctgaatttcaggtcatgaaggactagggacaccttgggagtcagaaagggtcattgggagcccgggctgatgcagacagacatcctcagctcccagacttcatggccagagatttataggatcc

SEQ ID NO: 6 - protein, 126 aa, Mus musculus
GVHSDVQLQESGPGLVKPSQTLSLTCTVSGYSMTSDYAWNWIRQFPGKGLEWIGYISYSGSTTYHPSVKSRITISRDTSKNQFFLQMNSVTTEDTATTYCTTIINYDKYDWYFDYWGQGTTVTVSS

SEQ ID NO: 7 - DNA, 630 bp, Mus musculus
  misc_feature (559)..(559): n is a, c, g, or t
agcttatgaatatgcaaatcctctgaatctacatggtaaatataggtttgtctataccacaaacagaaaaacatgagatacagttctctctacagttactgagcacacaggacctcaccatgggatggagctgtatcatcctcttcttggtagcaacagctacaggtaaggggctcacagtagcaggcttgaggtctggacatatatatgggtgacaatgacatccactttgcctttctctccacaggtgtccactccgacattgtgctgacacagtctcctgcttccttagttgtgtctgtgaggcagagggccaccatctcatgcagggccagccaaagtgtcaggacatcaagttatagttatatacactggtaccaacagaaaccaggacagccacccaaactcctcatctactatgcatccagcctagaatctggggtccctgccaggttcagtggcagtgggtctgggacagacttcaccctcaacatcagtcctgtggaggaggaggatactgcaacatattactgtcagcacagttgggagattccgtggncgttcggtccaggcaccaaagtggaaatcaaacgtgagtagaatttaaactttgcttcctcagttggatcc

SEQ ID NO: 8 - protein, 114 aa, Mus musculus
  misc_feature (104)..(104): Xaa can be any naturally occurring amino acid
GVHSDIVLTQSPASLVVSVRQRATISCRASQSVRTSSYSYIHWYQQKPGQPPKLLIYYASSLESGVPARFSGSGSGTFTLNISPVEEEDTATYYCQHSWEIPWXFGPGTKVEIK

SEQ ID NO: 9 - protein, 122 aa, Mus musculus
DVQLQESGPGLVKPSQTLSLTCTVSGYSMTSDYAWNWIRQFPGKGLEWIGYISYSGSTSYHPSVKSRITISRDTSKNQFFLQMNSVTTEDTATYYCTTIINYDKYDWYFDVWGQGTTVTVSS

SEQ ID NO: 10 - protein, 122 aa, Mus musculus
DVQLQESGPGLVKPSQTLSLTCTVSGYSMTSDYAWNWIRQFPGKGLEWIGYISYSGSTSYHPSLKSRISISRDTSKNQFFLQMNSVTTEDTATYYCTTIINYDKYDWYFDVWGQGTTVTVSS

SEQ ID NO: 11 - protein, 122 aa, Mus musculus
DVQLQESGPGLVKPSQTLSLTCTVSGYSITSDYAWNWIRQFPGKGLEWIGYISYSGSTSYHPSLKSRISISRDTSKNQFFLQMNSVTTEDTATYYCTTIINYDKYDWYFDVWGQGTTVTVSS

SEQ ID NO: 12 - protein, 122 aa, Mus musculus
DVQLQESGPGLVKPSQTLSLTCTVTGYSITSDYAWNWIRQFPGKGLEWMGYISYSGSTSYHPSLKSRISISRDTSKNQFFLQMNSVTTEDTATYYCTTIINYDKYDWYFDVWGQGTTVTVSS

SEQ ID NO: 13 - protein, 111 aa, Mus musculus
DIVLTQSPASLVVSVRQRATISCRASQSVRTSSYSYIHWYQQKPGQPPKLLIYYASSLESGVPARFSGSGSGTDFTLNISPVEEEDTATYYCQHSWEIPWTFGPGTKVEIK

SEQ ID NO: 14 - protein, 111 aa, Mus musculus
DIVLTQSPASLVVSVRQRATISCRASQSVRTSSYSYIHWYQQKPGQPPKLLIKYASSLESGVPARFSGSGSGTDFTLNISPVEEEDTATYYCQHSWEIPWTFGPGTTVEIK

(19) United States
(12) Patent Application Publication
(10) Pub. No.: US 2007/0224196 A1
(43) Pub. Date: Sep. 27, 2007

(54) Immunogenicity-reduced anti-cr1 antibody and compositions and methods of treatment based thereon

(75) Inventors: Jeff Himawan, San Jose, CA (US); Anita A. Hamilton, Aberdeen (GB); Francis J. Carr, Cambridge (GB)

Correspondence Address: LAHIVE & COCKFIELD, LLP, ONE POST OFFICE SQUARE, BOSTON, MA, 02109-2127, US

(73) Assignee: EluSys Therapeutics, Inc., Pine Brook, NJ (US)

(21) Appl. No.: 10/551,525

(22) PCT Filed: Mar. 29, 2004
(86) PCT No.: PCT/US04/09622

§ 371 Date: Jan. 8, 2007

Related U.S. Application Data
(60) Provisional application No. 60/458,869, filed on Mar. 28, 2003.

(51) Int. Cl. A61K 48/00; A61K 31/70; A61K 39/395; C12N 5/06; C07K 16/28

(52) U.S. Cl. 424/144.1; 530/388.22; 435/334; 514/44

(57) ABSTRACT

The invention provides immunogenicity-reduced antibodies or antibody fragments that bind a human CR1 receptor. The immunogenicity-reduced anti-CR1 antibody of the invention comprises one or more non-human sequences modified to comprise one or more amino acid substitutions so that the immunogenicity-reduced antibody id non-immunogenic or less immunogenic to a human. The invention also provides bispecific molecules comprising such an immunogenicity-reduced anti-CR1 antibody and an antigen-recognition portion that binds a pathogen. The invention further provides methods and compositions for the treatment of diseases or disorders caused by a blood-borne immunogenic pathogen using the bispecific molecule the invention of the invention.

This application claims the benefit under 35 U.S.C. § 119(e) of U.S. Provisional Patent Application No. 60/458,869, filed on Mar. 28, 2003, which is incorporated herein by reference in its entirety.

### 1. FIELD OF THE INVENTION

The invention relates to immunogenicity-reduced antibodies or antibody fragments that bind a human CR1 receptor. The immunogenicity-reduced anti-CR1 antibody of the invention comprises one or more non-human sequences modified to comprise one or more amino acid substitutions so that the immunogenicity-reduced antibody is non-immunogenic or less immunogenic to a human. The invention also relates to bispecific molecules comprising such an immunogenicity-reduced anti-CR1 antibody and an antigen-recognition portion that binds a pathogen. The invention further relates to methods and compositions for the treatment of diseases or disorders caused by a blood-borne immunogenic pathogen using the bispecific molecule of the invention.

### 2. BACKGROUND OF THE INVENTION

Primate erythrocytes, or red blood cells (RBC's), play an essential role in the clearance of antigens from the circulatory system. The formation of an immune complex in the circulatory system activates the complement factor C3b in primates and leads to the binding of C3b to the immune complex. The C3b/immune complex then binds to the type 1 complement receptor (CR1), a C3b receptor, expressed on the surface of erythrocytes via the C3b molecule attached to the immune complex. The immune complex is then chaperoned by the erythrocyte to the reticuloendothelial system (RES) in the liver and spleen for desctruction. The RES cells, most notably the fixed-tissue macrophages in the liver called Kupffer cells, recognize the erythrocyte bound immune complex and remove this complex from the RBC by severing the C3b receptor-RBC junction, producing a liberated erythrocyte and a C3b receptor/immune complex which is then engulfed by the Kupffer cells and is completely destroyed within subcellular organelles of the Kupffer cells.

This pathogen clearance process has been shown to be involved in the clearance of both microorganisms and soluble pathogens. For example, bacteria opsonized with both antibodies (Abs) and complement adhere to erythrocytes and this binding leads to enhanced phagocytosis and killing of the micro-organisms. It has also been shown that in some instances a soluble antibody (Ab)-protein antigen (Ag) immune complex (nonparticulate) that form in the circulation can fix complement, bind to erythrocytes, and then be cleared from the circulation and destroyed in the liver and spleen (Schifferli et al., 1989, Kidney Int. 35:993, Cornacoff et al., 1983, J. Clin. Invest. 71:236, Hebert et al., 1987, Kidney Int. 31:877). This pathogen clearance process, however, is complement-dependent, i.e., confined to immune complexes recognized by the C3b receptor, and is ineffective in removing immune complexes which are not recognized by the C3b receptor.

Taylor et al. have discovered a complement independent method of removing pathogens from the circulatory system. Taylor et al. have shown that chemical crosslinking of a first monoclonal antibody (mAb) specific to a primate C3b receptor to a second monoclonal antibody specific to a pathogenic molecule creates a bispecific heteropolymeric antibody (HP) which offers a mechanism for binding a pathogenic molecule to a primate's C3b receptor without complement activation (U.S. Pat. Nos. 5,487,890; 5,470,570; and 5,879,679). Taylor also reported a HP which can be used to remove a pathogenic antigen specific autoantibody from the circulation. Such a HP, also referred to as an “Antigen-based Heteropolymer” (AHP), contains a CR1 specific monoclonal antibody cross-linked to an antigen (see, e.g., U.S. Pat. No. 5,879,679; Lindorfer, et al., 2001, Immunol. Rev. 183: 10-24; Lindorfer, et al., 2001, J Immunol Methods 248: 125-138; Ferguson, et. al., 1995, Arthritis Rheum 38: 190-200).

In addition to HP and AHP produced by cross-linking, bispecific molecules that have a first antigen recognition domain which binds a C3b-like receptor, e.g., a complement receptor 1 (CR1), and a second antigen recognition domain which binds an antigen can also be produced by methods that do not involve chemical cross-linking (see, e.g., PCT publication WO 02/46208; and PCT publication WO 01/80883). PCT publication WO 01/80833 describes bispecific antibodies produced by methods involving fusion of hybridoma cell lines, recombinant techniques, and in vitro reconstitution of heavy and light chains obtained from appropriate monoclonal antibodies. PCT publication WO 02/46208 describes bispecific molecules produced by protein trans-splicing.

Kuhn et al. (1998, J. Immunol. 160: 5088-5097) discloses a method to bind target pathogens (both micro-organisms and protein antigens) to primate erythrocytes via CR1 with a very high level of efficiency in the complete absence of complement (Taylor et al., 1991, Proc. Natl. Acad. Sci. USA 88:3305; Powers et al., 1995, Infect. Immun. 63:1329; Reist et al., 1994, Eur. J. Immunol. 24:2018; Taylor et al., 1995, J. Hematother. 4:357). The method is based on using bispecific monoclonal antibody (mAb) complexes that are constructed by cross-linking a monoclonal antibody specific for CR1 (which serves as a surrogate for C3b) with a monoclonal antibody specific for the target pathogen. Based on Nelson's original work and the more widely studied erythrocyte-based immune complex clearance phenomenon, these bispecific complexes (heteropolymers (HP); anti-CR1 monoclonal antibody x anti-pathogen monoclonal antibody) are believed to have the potential to bind both soluble and particulate pathogens to erythrocytes in the bloodstream and then to present the pathogens to acceptor cells for phagocytosis and destruction. Kuhn et al. (1998, J. Immunol. 160: 5088-5097) also discloses that in vivo experiments in monkey models have verified that once bound to erythrocyte CR1 via specific heteropolymers, both soluble proteins and a prototype virus are cleared from the circulation and destroyed in the liver by a mechanism quite similar, in many respects, to the erythrocyte-immune complex clearance reaction (Reist et al., 1994, Eur. J. Immunol. 24:2018; Ferguson et al., 1995, J. Immunol. 155:339; Taylor et al., 1997, J. Immunol. 158:842 (abstract)).

Kuhn et al. (1998, J. Immunol. 160: 5088-5097) also discloses the use of an in vitro model, similar to that examined by Nelson, which uses E. coli as a model particulate pathogen. Specific heteropolymers were used to bind E. coli to primate erythrocytes, and the transfer of this erythrocyte-bound substrate to human monocytes was examined. The results of these studies, performed in the absence of complement, indicated that E. coli bound to erythrocyte CR1 via heteropolymers are indeed phagocytosed and destroyed by human monocytes. Kuhn et al. also discloses that this transfer reaction, which includes the concomitant loss of erythrocyte CR1, shows a striking similarity to the in vivo reaction by which substrates bound to erythrocyte CR1 are cleared from the circulation in primates.

Lindorfer et al. (2001, J. Immunol. 167(4):2240-9) discloses a bispecific heteropolymer, consisting of a mAb specific for the primate CR1 cross-linked with an anti-bacterial mAb, to target bacteria in the bloodstream in an acute infusion model in monkeys. In vitro studies demonstrated a variable level of complement-mediated binding (immune adherence) of Pseudomonas aeruginosa (strain PAO1) to primate erythrocytes in serum. In vivo experiments in animals depleted of complement revealed that binding of bacteria to erythrocytes was <1% before administration of the bispecific heteropolymer, but within 5 min of its infusion, >99% of the bacteria bound to the erythrocytes. In complement-replete monkeys, a variable fraction of infused bacteria bound to erythrocytes. Treatment of these complement-replete monkeys with the bispecific heteropolymer led to >99% binding of bacteria to erythrocytes. Twenty-four-hour survival studies were conducted; several clinical parameters, including the degree of lung damage, cytokine levels, and liver enzymes in the circulation, indicated that the bispecific heteropolymer provided a degree of protection against the bacterial challenge.

Lindorfer et al. (Immunological Review, 2001, 183:10-24) reported HP constructs using some of the neutralizing murine antibodies specific for the surface E glycoprotein of dengue virus. Such HP constructs can bind and clear dengus virus from the circulation of the animal model tested.

In the above-described methods, the bispecific heteropolymer comprises a murine anti-CR1 monoclonal antibody. When administered to a human patient, the murine anti-CR1 monoclonal antibody may elicit an immune response in the patient by eliciting the production of human anti-murine antibodies (HAMA). The patient's anti-murine antibodies may bind and clear the bispecific heteropolymer. The patient may also develop an allergic sensitivity to the murine antibody and be at risk of anaphylactic shock upon any future exposure to murine antibodies.

To reduce the immunogenicity of non-human antibodies, techniques have been developed to modify an antibody of non-human origin by introducing sequences that are present in human antibodies, while retaining particular single amino acid residues at positions critical to maintaining the antibody's binding specificity and affinity. For example, chimeric antibodies, which are antibody molecules in which different portions are derived from different animal species, such as those having a variable region derived from a murine mAb and a human immunoglobulin constant region (Morrison, et al., 1984, Proc. Natl. Acad. Sci., 81, 6851-6855; Neuberger, et al., 1984, Nature 312, 604-608; Takeda, et al., 1985, Nature, 314, 452-454; Cabilly et al., U.S. Pat. No. 4,816,567; and Boss et al., U.S. Pat. No. 4,816,397) can be produced by splicing the genes from a mouse antibody molecule of appropriate antigen specificity together with genes from a human antibody molecule of appropriate biological activity can be used. Humanized antibodies, which are antibody molecules from non-human species having one or more complementarity determining regions (CDRs) from the non-human species and a framework region from a human immunoglobulin molecule, are also developed (see e.g., U.S. Pat. No. 5,585,089, which is incorporated herein by reference in its entirety.). Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Techniques for elimination of T cell epitopes from proteins such as antibodies has also been disclosed (see WO 00/34317 and WO 98/52976). In these techniques, potential T cell epitopes in a protein are first identified, and the identified epitopes are then removed by modifying the amino acids sequences.

There is therefore a need for a non-immunogenic or less immunogeic antibody that can be administered to a human patient without eliciting an immune response.

Discussion or citation of a reference herein shall not be construed as an admission that such reference is prior art to the present invention.

### 3. SUMMARY OF THE INVENTION

The present invention provides methods and compositions for rapidly and efficiently clearing an antigen of interest from the circulation. The molecules of the invention utilize the unique properties of CR1, expressed on the surface of hematopoietic cells in humans, to clear circulating antigens or pathogens. In particular, the compositions of the invention are useful for rapidly and efficiently clearing antigens from the circulation. The invention provides proteins encoded by and nucleotide sequences of immunogenicity-reduced anti-CR1 antibody genes. The invention further provides fragments and other derivatives and analogs of such immunogenicity-reduced anti-CR1 antibody proteins. Nucleic acids encoding such fragments or derivatives are also within the scope of the invention. Production of the foregoing proteins, e.g., by recombinant methods, is provided.

The invention also provides proteins and derivatives of immunogenicity-reduced anti-CR1 antibodies, including fusion/chimeric proteins that are functionally active, i.e., that are capable of displaying binding to CR1.

The immunogenicity-reduced anti-CR1 molecules of the invention, e.g., antibodies, derivatives and/or fragments thereof, have binding specificity for CR1. In preferred embodiments, immunogenicity-reduced anti-CR1 molecules of the invention can be used to make a bispecific molecule or heteropolymer. In certain embodiments, the heteropolymer is a bispecific antibody. The bispecific antibody has a first binding domain that binds to an antigen present in the circulation of a mammal and a second binding domain that binds to complement receptor 1 (CR1) (also known as CD35 in primates). In another embodiment, the invention provides immunogenicity-reduced molecules that utilize the unique properties of CR1, expressed on the surface of hematopoietic cells, to rapidly and efficiently clear an antigen of interest from the circulation.

The invention also provides methods of making anti-CR1 immunogenicity-reduced heteropolymers or bispecific antibodies, as well as therapeutic and prophylactic uses thereof, as well as to kits containing the anti-CR1 immunogenicity-reduced heteropolymers or bispecific antibodies, nucleic acids encoding bispecific molecules that are polypeptides, and cells transformed with the nucleic acids, and recombinant methods of production of the bispecific molecules.

The invention further provides a method for the treatment or prevention of diseases or disorders caused by a blood-borne immunogenic pathogen in a subject comprising administering to the subject, in an amount effective for said treatment or prevention, an immunogenicity-reduced bispecific antibody that immunospecifically binds CR1 and an antigen of interest. In certain embodiments, the antigen of interest is an antigen of a pathogen, an autoantigen or a blood-borne protein desired to be removed from the circulatory system of a mammal.

The invention yet further provides a method for identifying an immunogenicity-reduced anti-CR1 antibody useful for clearance of an antigen of interest from the circulation, comprising determining whether administration of the immunogenicity-reduced anti-CR1 antibody leads to clearance of the antigen of interest from the circulation. In preferred embodiments, the immunogenicity-reduced anti-CR1 antibody is a bispecific antibody or derivative thereof.

The invention further provides isolated nucleic acids encoding an immunogenicity-reduced antibody that competes for binding to CR1 with human complement. The invention further provides methods of isolating-nucleic acids encoding immunogenicity-reduced antibodies that immunospecifically bind CR1.

The invention also provides kits containing anti-CR1 immunogenicity-reduced heteropolymers or bispecific antibodies, nucleic acids encoding bispecific molecules that are polypeptides, and cells transformed with the nucleic acids, and recombinant methods of production of the bispecific molecules.

### 4. BRIEF DESCRIPTION OF FIGURES

FIG. 1. DNA [SEQ ID NO: 1] and amino acid [SEQ ID NO: 2] sequences of murine E11 V_{H}. For details, see Section 6 (Example 1).

FIG. 2. DNA [SEQ ID NO: 3] and amino acid [SEQ ID NO: 4] sequence of murine E11 V_{L}. For details, see Section 6 (Example 1).

FIG. 3. DNA [SEQ ID NO: 5] and amino acid [SEQ ID NO: 6] sequence of primary immunogenicity-reduced E11 heavy chain, E DIVHv1. For details, see Section 6 (Example 1).

FIG. 4. DNA [SEQ ID NO: 7] and amino acid [SEQ ID NO: 8] sequence of primary immunogenicity-reduced E11 light chain, E DIVLv1. For details, see Section 6 (Example 1).

FIG. 5. Comparison of amino acid sequences of murine and immunogenicity-reduced E V_{H}. For details, see Section 6 (Example 1). Murine E11 V_{H}: MoVH.PRO, SEQ ID NO:2; immunogenicity-reduced E11 V_{H} v1: DiVH-v1.PRO, SEQ ID NO. 6; immunogenicity-reduced E11 V_{H} v2: DiVH-v2.PRO, SEQ ID NO. 9; immunogenicity-reduced E11 V_{H} v3: DiVH-v3.PRO, SEQ ID NO. 10; immunogenicity-reduced E11 V_{H} v4: DiVH-v4.PRO, SEQ ID NO. 11; immunogenicity-reduced E11 V_{H} v5: DiVH-v5.PRO, SEQ ID NO. 12.

FIG. 6. Comparison of amino acid sequences of murine and immunogenicity-reduced E V_{L}. For details, see Section 6 (Example 1). Murine E11 V_{L}: MoVL.PRO, SEQ ID NO:8; immunogenicity-reduced E11 V_{L} v1: DiVL-v1.PRO, SEQ ID NO. 13; immunogenicity-reduced E11 V_{L} v2: DiVL-v2.PRO, SEQ ID NO. 14.

FIG. 7. Heavy chain expression vector. For details, see Section 6 (Example 1).

FIG. 8. Light chain expression vector. For details, see Section 6 (Example 1).

FIG. 9. Binding of murine and chimeric E11 antibodies. For details, see Section 6 (Example 1).

FIG. 10. Binding of immunogenicity-reduced antibodies E DI VH5/VL2 and E DI VH3/VL2 compared with the binding of a chimeric antibody (“E chimaeric Ab”). For details, see Section 6 (Example 1).

FIG. 11. Binding of immunogenicity-reduced antibodies E DI VH4/VL1 and E DI VH2/VL1 compared with the binding of a chimeric antibody (“E chimaeric Ab”). For details, see Section 6 (Example 1).

FIG. 12. Binding of immunogenicity-reduced antibodies E DI VH1/VL1, E DI VH1/VL2 and E DI VH3/VL1 compared with the binding of a chimeric antibody (“E chimaeric Ab”). For details, see Section 6 (Example 1).

FIG. 13. Binding of immunogenicity-reduced antibodies E DI VH5/VL1 and E DI VH4/VL2 compared with the binding of a chimeric antibody (“E chimaeric Ab”). For details, see Section 6 (Example 1).

FIGS. 14A-B Macrophage viability assay showed that a bispecific molecule, 3F3 cross-linked to 19E9, protected macrophages from the lethal toxin (containing PA and LF) of B. anthracis in the presence of erythrocytes.

### 5. DETAILED DESCRIPTION OF THE INVENTION

The present invention provides immunogenicity-reduced antibodies that bind a human CR1 receptor. As used herein, the term “immunogenicity-reduced antibody” refers to an antibody that is of a non-human origin but has been modified, i.e., with one or more amino acid substitutions, so that it is non-immunogenic or less immunogenic to a human when compared to the starting non-human antibody. The present invention also provides immunogenicity-reduced bispecific molecules that comprise an immunogenicity-reduced anti-CR1 antibody and a second antigen-binding portion which bind a pathogenic antigenic molecule.

The immunoglobulin molecules are encoded by genes which include the kappa, lambda, alpha, gamma, delta, epsilon and mu constant regions, as well as a myriad of immunoglobulin variable regions. Light chains are classified as either kappa or lambda. Light chains comprise a variable light (V_{L}) and a constant light (C_{L}) domain. Heavy chains are classified as gamma, mu, alpha, delta, or epsilon, which in turn define the immunoglobulin classes IgG, IgM, IgA, IgD and IgE, respectively. Heavy chains comprise variable heavy (V_{H}), constant heavy 1 (CH1), hinge, constant heavy 2 (CH2), and constant heavy 3 (CH3) domains. The IgG heavy chains are further sub-classified based on their sequence variation, and the subclasses are designated IgG1, IgG2, IgG3 and IgG4.

Antibodies can be further broken down into two pairs of a light and heavy domain. The paired V_{L} and V_{H} domains each comprise a series of seven subdomains: framework region 1 (FR1), complementarity determining region 1 (CDR1), framework region 2 (FR2), complementarity determining region 2 (CDR2), framework region 3 (FR3), complementarity determining region 3 (CDR3), framework region 4 (FR4) which constitute the antibody-antigen recognition domain.

The present invention provides methods and compositions for rapidly and efficiently clearing an antigen of interest from the circulation. The molecules of the invention utilize the unique properties of CR1, expressed on the surface of hematopoietic cells in humans, to clear circulating antigens. In particular, the compositions of the invention are useful for rapidly and efficiently clearing antigens from the circulation. The invention provides proteins encoded by and nucleotide sequences of immunogenicity-reduced anti-CR1 antibody genes. The invention further provides fragments and other derivatives and analogs of such immunogenicity-reduced anti-CR1 antibody proteins. Nucleic acids encoding such fragments or derivatives are also within the scope of the invention. Production of the foregoing proteins, e.g., by recombinant methods, is provided.

Wherein the protein of the invention is an immunogenicity-reduced antibody or derivative thereof, the antibody or derivative is preferably a monoclonal antibody, more preferably a recombinant antibody, and most preferably is human, humanized, or chimeric. immunogenicity-reduced antibodies to CR1 encompassed by the invention include human, chimeric, humanized antibodies. In one embodiment, an anti-CR1 immunogenicity-reduced antibody or derivative thereof is a bispecific molecule.

The immunogenicity-reduced antibodies of the invention should be poorly recognized as foreign proteins by the human immune system, that is, they are poorly immunogenic, thus making them preferable for therapeutic or diagnostic use in humans. In particular, a human immune reaction would diminish the therapeutic effectiveness of immunogenicity-reduced bispecific antibodies with regard to repeated treatments.

The immunogenicity-reduced anti-CR1 molecules of the invention, e.g. antibodies, derivatives and/or fragments thereof, have binding specificity for CR1. In preferred embodiments, immunogenicity-reduced anti-CR1 molecules of the invention can be used to make a bispecific molecule or heteropolymer. In certain embodiments, the heteropolymer is a bispecific antibody. The bispecific antibody has a first binding domain that binds to an antigen present in the circulation of a human or primate and a second binding domain that binds to complement receptor 1 (CR1) (also known as CD35 in primates). In another embodiment, the invention provides immunogenicity-reduced molecules that utilize the unique properties of the CR1 receptor (for example, CR1 on erythrocytes in humans), expressed on the surface of hematopoietic cells, to rapidly and efficiently clear an antigen of interest from the circulation.

The invention also provides proteins and derivatives of immunogenicity-reduced anti-CR1 antibodies, including fusion/chimeric proteins that are functionally active, i.e., that are capable of displaying binding to CR1.

The invention also provides methods of making anti-CR1 immunogenicity-reduced heteropolymers or bispecific antibodies, as well as therapeutic and prophylactic uses thereof, as well as to kits containing the anti-CR1 immunogenicity-reduced heteropolymers or bispecific antibodies, nucleic acids encoding bispecific molecules that are polypeptides, and cells transformed with the nucleic acids, and recombinant methods of production of the bispecific molecules.

The invention further provides a method for the treatment or prevention of diseases or disorders caused by a blood-borne immunogenic pathogen in a subject comprising administering to the subject, in an amount effective for said treatment or prevention, an immunogenicity-reduced bispecific antibody that specifically binds CR1 and an antigen of interest. In certain embodiments, the antigen of interest is an antigen of a pathogen, an autoantigen or a blood-borne protein desired to be removed from the circulatory system of a human or primate.

The compositions and methods of the invention are useful for the treatment of diseases, disorders, or other conditions wherein an antigenic molecule is desired to be removed from the circulation (i e., where the antigenic molecule is, or is a component of, a causative agent of the condition), as well as for the prevention of the onset of the symptoms and signs of such conditions, or for the delay of the symptoms and signs in the evolution of these conditions. The methods of the invention will be, for example, useful for the treatment of such conditions, including the improvement or alleviation of any symptoms and signs of such conditions, the improvement of any pathological or laboratory findings of such conditions, the delay of the evolution of such conditions, the delay of onset of any symptoms and signs of such conditions, as well as the prevention of occurrence of such conditions, and the prevention of the onset of any of the symptoms and signs of such conditions.

The invention further provides isolated nucleic acids encoding an immunogenicity-reduced antibody that competes for binding to CR1 with human complement. The invention further provides methods of isolating nucleic acids encoding immunogenicity-reduced antibodies that immunospecifically bind CR1.

The C3b receptor is known as the complement receptor 1 (CR1) in primates or CD35. As used herein, the term “CR1 receptor” is understood to mean any mammalian circulatory molecule that has an analogous function to a primate CR1 receptor. According to the invention, CR1 molecules bind to complement opsonized immune complexes in the blood stream and carry them to the liver and spleen, where they are destroyed. The red blood cells are returned to circulation.

Blood-borne antigens that may be bound by the molecules of the invention include, but are not limited to, an antigen of a pathogen, an autoantigen or a blood-borne protein desired to be removed from the circulatory system of a mammal. In certain embodiments, the antigen of the pathogen (“pathogenic antigenic molecule”) is an antigen of an infectious agent, including but not limited to, a microbial antigen, e.g., viral, bacterial, fungal, or yeast antigen; or a protozoan or parasite antigen. In other embodiments, the pathogenic antigenic molecule may be a drug, toxin or a low density lipoprotein.

As used herein, the term “epitope” refers to an antigenic determinant, i.e., a region of a molecule that provokes an immunological response in a host or is bound by an antibody. This region can but need not comprise consecutive amino acids. The term epitope is also known in the art as “antigenic determinant.” An epitope may comprise as few as three amino acids in a spatial conformation that is unique to the immune system of the host. Generally, an epitope consists of at least five such amino acids, and more usually consists of at least 8-10 such amino acids. Methods for determining the spatial conformation of such amino acids are known in the art.

The invention also provides methods and compositions that can be used in conjunction with radiolabeled antibodies, which are used in detection of an antigen of interest in the circulation, e.g., a bacterial-, viral-, or parasite-derived antigen. An inmunogenicity-reduced bispecific anti-CR1 antibody can be radiolabeled to detect a bacterial-, viral-, or parasite-derived antigen in the circulation, e.g., radiolabeled antibodies can be injected to a host and then visualized by any imaging methods that detects specifically the radiation site(s) known in the art.

As used herein, the term “radiolabeled antibody” refers to antibodies that are linked with radioactive markers, such as indium-111 (¹¹¹In). (See Hagan P. L. et al., 1985, J. Nucl. Med. 26:1418-1423).

In a preferred embodiment, the methods and compositions of the invention are used to treat a disease in a human or non-human primates. In another embodiment, the methods and compositions of the invention are used to treat a an infection, including but not limited to, a viral, bacterial, fungal, protozoan, or parasitic infection.

The methods provided by the invention enable the binding of any target antigen in the bloodstream to the surface of a red blood cell of the CR1 receptor without the need to activate the complement system. By completely bypassing the complement cascade, the methods of the invention significantly increase the ability of the target antigen to bind to the surface of the red blood cell, thus substantially increasing the efficiency with which immune adherence destroys the offending blood-borne pathogens.

The methods and compositions of the invention offer a significant advance in the management and treatment of a broad range of blood-borne diseases. The methods and compositions of the invention are advantageous because they enable the rapid, safe and efficient removal and destruction of blood-borne pathogens, such as viral particles, bacteria, toxins and autoantibodies, from the bloodstream by simply injecting a therapeutic compound into the bloodstream of a patient. The methods and compositions of the invention can be used to treat multiple scores of different diseases by producing an appropriate immunogenicity-reduced bispecific anti-CR1 antibody for each designated pathogen. Both the processes of manufacturing monoclonal antibodies and of joining two monoclonal antibodies to each other to form bispecific antibodies are well-known in the art. The compositions of the invention are able to remove and destroy members of the major classes of blood-borne pathogens, thus providing an effective treatment for a broad array of different diseases. The non-immunogenic, immunogenicity-reduced anti-CR1 antibody of the invention can be administered to a patient on multiple occasions over long time periods without inducing an immune response, can bind both soluble and particulate pathogens to erythrocytes in the bloodstream, and then present the pathogens to acceptor cells for phagocytosis and destruction.

For clarity of disclosure, and not by way of limitation, the detailed description of the invention is divided into the subsections that follow.

### 5.1 IMMUNOGENICITY-REDUCED ANTI-CR1 ANTIBODIES AND PRODUCTION

The invention provides immunogenicity-reduced antibodies or antibody fragments that bind a human CR1 receptor. The immunogenicity-reduced anti-CR1 antibodies of the invention can be any immunogenicity-reduced antibody that contains a CR1 binding domain and an effector domain. In some embodiments, the immunogenicity-reduced anti-CR1 antibody is an immunogenicity-reduced anti-CR1 monoclonal antibody (mAb). In some embodiments, the constant regions of the immunogenicity-reduced anti-CR1 antibody are human. In preferred embodiments, the immunogenicity-reduced anti-CR1 antibody comprises one or more non-human V_{H} or V_{L} sequences modified to comprise one or more amino acid substitutions so that the immunogenicity-reduced antibody is non-immunogenic or less immunogenic to a human when compared to the respective unmodified non-human sequences (see WO 00/34317 and WO 98/52976).

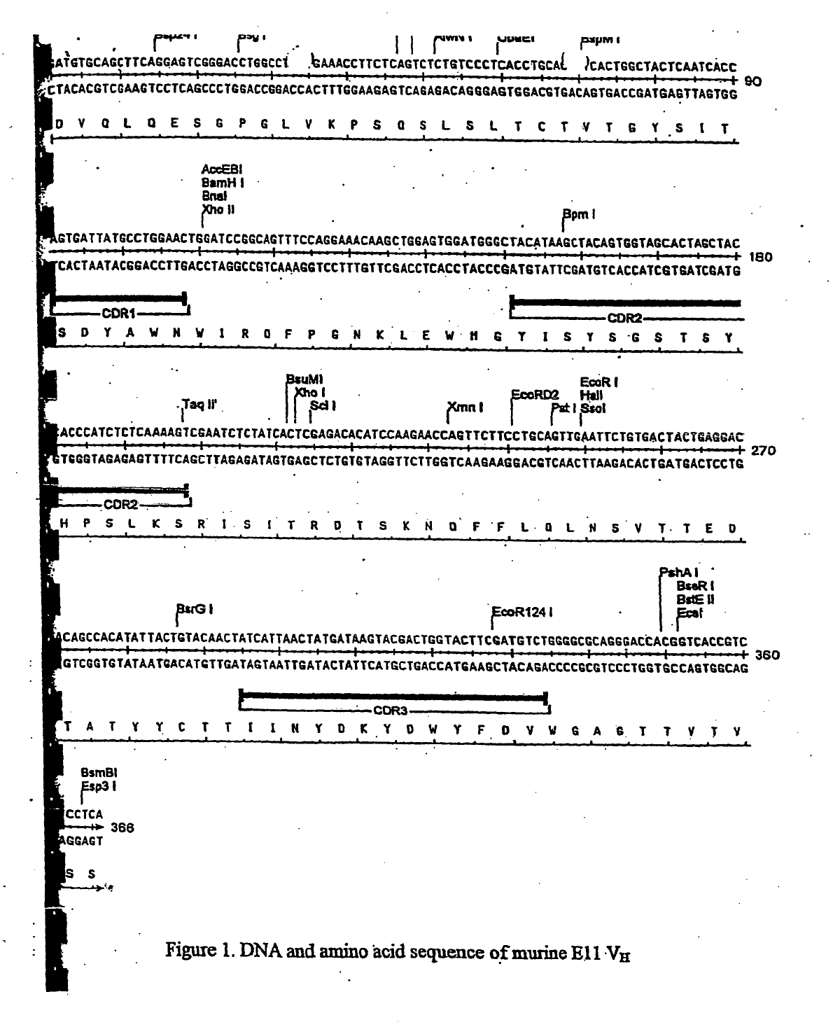

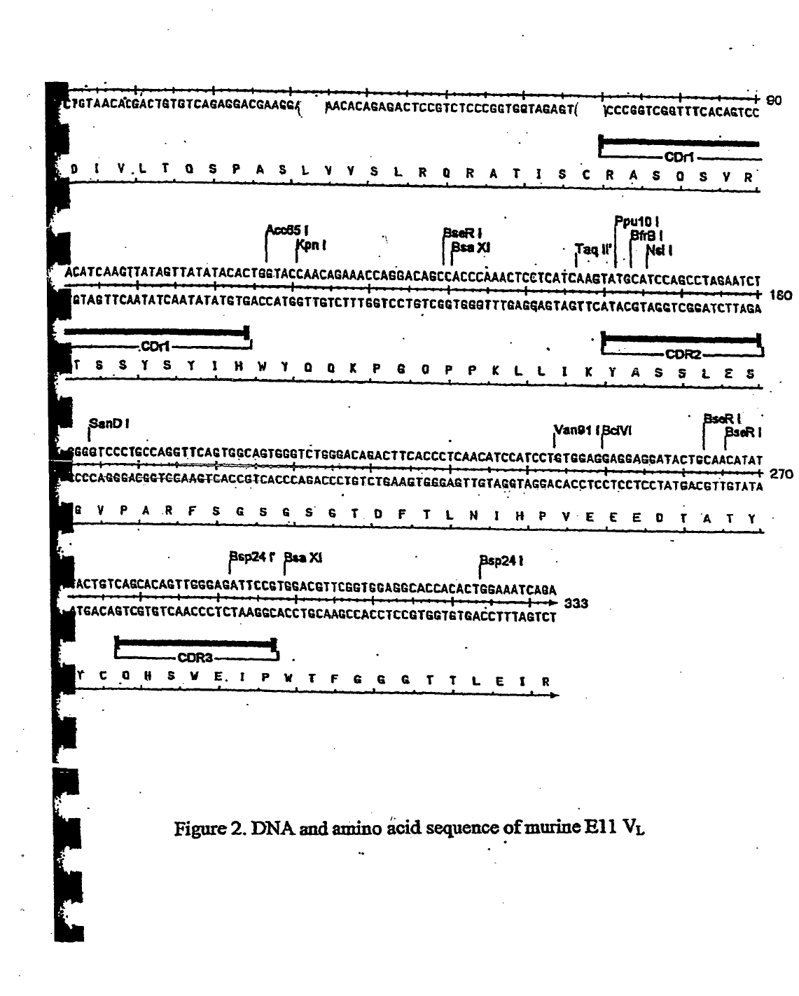

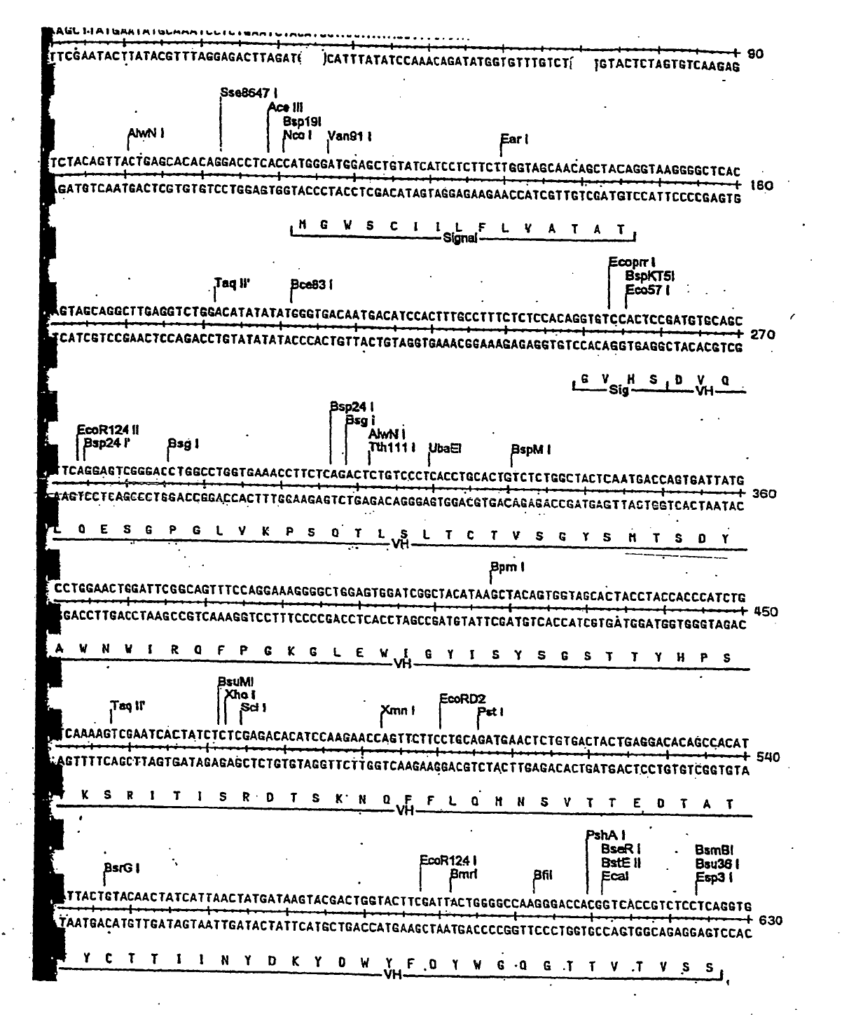

In preferred embodiments, the immunogenicity-reduced anti-CR1 antibody comprises one or more non-human V_{H} or V_{L} sequences, in each of which one or more human T cell epitopes are modified by substitution of one or more amino acids. In preferred embodiments, the invention provides such immunogenicity-reduced V_{H} or V_{L} sequences generated from a murine V_{H} or V_{L} sequences. In a preferred embodiment, the immunogenicity-reduced V_{H} or V_{L} sequences are generated from the murine V_{H} and V_{L} sequences that are obtained from an anti-CR1 antibody produced by murine E11 hybridoma (Catalog# 184-020, Ancell Immunology Research Products MN; N. Hogg et al., 1984, Eur J Immunol 14: 236-243; and Leukocyte Typing IV, W. Knapp, et al, eds., Oxford University Press, Oxford, 1989, p. 829-830). The DNA (SEQ ID NO: 1) and amino acid (SEQ ID NO: 2) sequences of murine E11 V_{H} is shown in FIG. 1. The DNA (SEQ ID NO: 3) and amino acid (SEQ ID NO: 4) sequence of murine E11 V_{L} is shown in FIG. 2.

In preferred embodiments, the invention provides a deimmunised molecule that specifically binds CR1 and comprises an immunogenicity-reduced V_{H} sequence which is the amino acid sequence as described by SEQ ID NO: 2, but with one or more of the following amino acid substitutions in SEQ ID NO: 2:

Position 17: Ser→Thr;

Position 25: Thr→Ser;

Position 29: Ile→Met;

Position 44: Asn→Lys;

Position 45: Lys→Gly;

Position 49: Met→Ile;

Position 59: Ser→Thr;

Position 64: Leu→Val;

Position 69: Ser→Thr;

Position 71: Thr→Ser;

Position 83: Leu→Met;

Position 111: Val→Tyr; and

Position 114: Ala→Gln.

In a preferred embodiment, the immunogenicity-reduced V_{H} sequence is with all the above identified amino acid substitutions (identified as VH1). In another preferred embodiment, the immunogenicity-reduced V_{H} sequence is with all the above identified amino acid substitutions except the substitutions at positions 59 and 111 (identified as VH2). In still another preferred embodiment, the immunogenicity-reduced V_{H} sequence is with all the above identified amino acid substitutions except the substitutions at positions 59, 64, 69, and 111 (identified as VH3). In still another preferred embodiment, the immunogenicity-reduced V_{H} sequence is with all the above identified amino acid substitutions except the substitutions at positions 29, 59, 64, 69, and 111 (identified as VH4). In still another preferred embodiment, the immunogenicity-reduced V_{H} sequence is with only 43, 44, 71, 83, and 114 of the above identified amino acid substitutions (identified as VH5).

In another embodiment, the invention provides an immunogenicity-reduced molecule that specifically binds CR1 and comprises an amino acid sequence as described by amino acid numbers 51-66 of SEQ ID NO: 2 (the complementarity determining region 2 (CDR2)) but with one or more of the following amino acid substitutions in SEQ ID NO: 2:

Position 59: Ser→Thr; and

Position 64: Leu→Val.

In another embodiment, the invention provides an immunogenicity-reduced molecule that specifically binds CR1 and comprises an amino acid sequence as described by amino acid numbers 99-112 of SEQ ID NO: 2 (the complementarity determining region 3 (CDR3)), but with the following amino acid substitution in SEQ ID NO: 2:

Position 11: Val→Tyr.

In another embodiment, the invention provides an immunogenicity-reduced molecule that specifically binds CR1 and comprises:

(a) an amino acid sequence as described by amino acid numbers 31-36 of SEQ ID NO: 2 (the complementarity determining region 1 (CDR1));

(b) an amino acid sequence as described by amino acid numbers 51-66 of SEQ ID NO: 2 (the complementarity determining region 2 (CDR2)) but with one or more of the following amino acid substitutions in SEQ ID NO: 2:

Position 59: Ser→Thr, and

Position 64: Leu→Val; and

(c) amino acid numbers 99-112 of SEQ ID NO: 2 (the complementarity determining region 3 (CDR3)) but with the following amino acid substitution in SEQ ID NO: 2:

Position 111: Val→Tyr.

In another embodiment, the invention provides an immunogenicity-reduced molecule that specifically binds CR1 and comprises SEQ ID NO: 4, but with one or more of the following amino acid substitutions in SEQ ID NO: 4:

Position 15: Leu→Val;

Position 53: Lys→Tyr;

Position 80: His→Ser;

Position 104: Gly→Pro;

Position 107: Thr→Lys;

Position 108: Leu→Val; and

Position 111: Arg→Lys.

In a preferred embodiment, the immunogenicity-reduced V_{L} sequence is with all the above identified amino acid substitutions (identified as VL1). In another preferred embodiment, the immunogenicity-reduced V_{L} sequence is with all the above identified amino acid substitutions except the substitutions at positions 53 and 107 (identified as VL2).

The invention also provides plasmid DNAs encoding immunogenicity-reduced antibody V regions described above: pUC19 E DIVH1 comprising nucleic acid sequence encoding VH1, pUC19 E DIVH2 comprising nucleic acid sequence encoding VH2, pUC19 E DIVH3 comprising nucleic acid sequence encoding VH3, pUC19 E DIVH4 comprising nucleic acid sequence encoding VH4, pUC19 E DIVH5 comprising nucleic acid sequence encoding VH5, pUC19 E DIVL1 comprising nucleic acid sequence encoding VL1, and pUC19 E DIVL2 comprising nucleic acid sequence encoding VL2.

The invention also provides immunogenicity-reduced anti-CR1 antibodies comprising one or more of VH1-VH5 and one or more of VL1-VL2. Preferably, the immunogenicity-reduced anti-CR1 antibodies comprise a human constant region. In a preferred embodiment, the immunogenicity-reduced anti-CR1 monoclonal antibody is 19E9 which comprises immunogenicity-reduced VH4 and VL1, and which is deposited at ATCC. In another preferred embodiment, the immunogenicity-reduced anti-CR1 monoclonal antibody is 12H10 which comprises immunogenicity-reduced VH3 and VL1, and which is deposited at ATCC. In still another preferred embodiment, the imnunogenicity-reduced anti-CR1 monoclonal antibody is 15A12 which comprises immunogenicity-reduced VH3 and VL2, and which is deposited at ATCC. In still another preferred embodiment, the immunogenicity-reduced anti-CR1 monoclonal antibody is 44H1 which comprises immunogenicity-reduced VH2 and VL1, and which is deposited at ATCC. In still another preferred embodiment, the immunogenicity-reduced anti-CR1 monoclonal antibody is 31C11 which comprises immunogenicity-reduced VH5 and VL2, and which is deposited in ATCC.

The immunogenicity-reduced anti-CR1 antibody can also be a chimeric antibody, such as but is not limited to a humanized monoclonal antibody in which the complementarity determining regions are mouse, and the framework regions and constant regions are human. In a specific embodiment, the immunogenicity-reduced chimeric antibody is 3G4 which comprises E11 murine variable regions linked with human IgG1 constant regions, and which is deposited at ATCC.

The immunogenicity-reduced antibodies of the invention may be of any isotype, but is preferably human IgG1.

In other embodiments, the immunogenicity-reduced anti-CR1 antibody is an immunogenicity-reduced anti-CR1 polypeptide antibody, including but is not limited to, an immunogenicity-reduced anti-CR1 single-chain variable region fragment (scFv) fused to the N-terminus of an immunoglobulin Fc domain. As used herein, an antibody can also be a single-chain antibody (scFv), which generally comprises a fusion polypeptide consisting of a variable domain of a light chain fused via a polypeptide linker to the variable domain of a heavy chain. The scFv of the invention can comprise any of the above described immunogenicity-reduced V_{H} and V_{L} of the invention.

The immunogenicity-reduced anti-CR1 antibody can also be antibody fragments. Examples of immunologically active fragments of immunoglobulin molecules include scFv, F(ab) and F(ab′)2 fragments which can be generated by treating the antibody with an enzyme such as pepsin or papain. Antibodies exist for example, as intact immunoglobulins or can be cleaved into a number of well-characterized fragments produced by digestion with various peptidases, such as papain or pepsin. Pepsin digests an antibody below the disulfide linkages in the hinge region to produce a F(ab)′₂ fragment of the antibody which is a dimer of the Fab composed of a light chain joined to a V_{H}-C_{H}1 by a disulfide bond. The F(ab)′₂ may be reduced under mild conditions to break the disulfide linkage in the hinge region thereby converting the F(ab)′₂ dimer to a Fab′ monomer. The Fab′ monomer is essentially an Fab with part of the hinge region. See Paul, ed., 1993, Fundamental Immunology, Third Edition (New York: Raven Press), for a detailed description of epitopes, antibodies and antibody fragments. One of skill in the art will recognize that such Fab′ fragments may be synthesized de novo either chemically or using recombinant DNA technology. Thus, as used herein, the term antibody fragments includes antibody fragments produced by the modification of whole antibodies or those synthesized de novo. The antibody fragment of the invention can comprise any of the above described inmunogenicity-reduced V_{H} and V_{L} of the invention.

In a preferred embodiment, immunogenicity-reduced anti-CR1 antibodies are designed and produced according to the method described in PCT publications WO 00/34317 and WO 98/52976, which are incorporated herein by reference in their entirety. In the embodiment, cDNA encoding V_{H} and V_{L} of a chosen non-human anti-CR1 antibody, e.g., a murine anti-CR1 antibody, are used as the starting sequences. The cDNAs can be obtained using standard methods. Optionally, the V_{H} and V_{L} clones obtained can be screened for inserts of the expected size by standard method known in the art, e.g., by PCR, and the DNA sequence of selected clones determined by standard methods. The locations of the complementarity determining regions (CDRs) can be determined using standard methods with reference to other antibody sequences disclosed in Kabat et al. (1991).

The non-human starting V_{H} and V_{L} sequences are compared to directories of human germline antibody genes (Cox et al., 1994; Tomlinson et al., 1992). The closest match human germline genes are selected as reference for the immunogenicity-reduced V_{H} and V_{L}. The starting V region sequences obtained are then subjected to peptide threading to identify potential T-cell epitopes, through analysis of binding to a plurality of different human MHC class II allotypes. The sequences can also be analyzed for presence of known human T-cell binding peptides from a suitable database, e.g., The Walter and Eliza Hall Institute of Medical Research, Melbourne, Australia, World Wide Web site wehil.wehi.edu.au, using a suitable program, e.g., the program “Searcher.”

Primary immunogenicity-reduced V_{H} and V_{L} sequences are designed to retain various preferred non-human amino acids in the starting sequences. Preferably, as generation of the primary immunogenicity-reduced sequences requires a small number of amino acid substitutions that might affect the binding of the final immunogenicity-reduced molecule, a plurality of other variant V_{H} and V_{L} sequences are also designed.

The immunogenicity-reduced variable regions are constructed by the method of overlapping PCR recombination. The cloned non-human starting V_{H} and V_{L} genes are used as templates for mutagenesis of the framework regions to the required immunogenicity-reduced sequences. Sets of mutagenic primer pairs are synthesized encompassing the regions to be altered. In preferred embodiments, the vectors VH-PCR1 and VL-PCR1 (Riechmann et al., 1988) can be used as templates to introduce a 5′ flanking sequence, including the leader signal peptide, leader intron and the murine immunoglobulin promoter, and a 3′ flanking sequence, including the splice site and intron sequences. The immunogenicity-reduced V regions produced are then cloned into a suitable plasmid, e.g., pUC 19, and the entire DNA sequence is confirmed to be correct for each immunogenicity-reduced V_{H} and V_{L}.

The immunogenicity-reduced heavy and light chain V-region genes can be excised from the plasmids as appropriate restriction fragments, which include the non-human heavy chain immunoglobulin promoter, the leader signal peptide, leader intron, the V_{H} or V_{L} sequence and the splice site. These are transferred to suitable expression vectors which include human constant regions, e.g., IgG1 constant regions, and markers for selection in mammalian cells.

The heavy and light chain expression vectors are preferably co-transfected in a variety of combinations into a suitable cell line by electroporation. Colonies expressing the selection marker gene are selected. Production of human antibody by transfected cell clones can be measured by ELISA for human IgG. Cell lines secreting antibody are selected and expanded. The immunogenicity-reduced antibodies are purified using standard method known in the art.

The immunogenicity-reduced antibodies are preferably screened for their binding affinities to RBCs. In a preferred embodiment, a modified antigen binding assay is used, in which the antibodies are reacted with RBCs in solution and the cells are then fixed to 96-well plates with poly L-lysine and glutaraldehyde at the end of the assay, just prior to the addition of the substrate. Washed erythrocytes are added to dilutions of antibody in 96-well V-bottom plates. Bound antibody is detected with biotinylated anti-human antibody or an antibody that binds the starting non-human antibody, then visualized using avidin alkaline phosphatase according to standard methods.

In a preferred embodiment, immunogenicity-reduced anti-CR1 antibodies are designed and produced according to the method described in PCT publications WO 00/34317 and WO 98/52976, which are incorporated herein by reference in their entirety. In the embodiment, cDNA encoding V_{H} and V_{L} of a chosen non-human anti-CR1 antibody, e.g., a murine anti-CR1 antibody, are used as the starting sequences. The cDNAs can be obtained using standard methods. Optionally, the V_{H} and V_{L} clones obtained can be screened for inserts of the expected size by standard method known in the art, e.g., by PCR, and the DNA sequence of selected clones determined by standard methods. The locations of the complementarity determining regions (CDRs) can be determined using standard methods with reference to other antibody sequences disclosed in Kabat et al. (1991).

The non-human starting V_{H} and V_{L} sequences are compared to directories of human germline antibody genes (Cox et al., 1994; Tomlinson et al., 1992). The closest match human germline genes are selected as reference for the immunogenicity-reduced V_{H} and V_{L}. The starting V region sequences obtained are then subjected to peptide threading to identify potential T-cell epitopes, through analysis of binding to a plurality of different human MHC class II allotypes. The sequences can also be analyzed for presence of known human T-cell binding peptides from a suitable database, e.g., The Walter and Eliza Hall Institute of Medical Research, Melbourne, Australia, World Wide Web site wehil.wehi.edu.au, using a suitable program, e.g., program “searcher.”

Primary immunogenicity-reduced V_{H} and V_{L} sequences are designed to retain various preferred non-human amino acids in the starting sequences. Preferably, as generation of the primary immunogenicity-reduced sequences requires a small number of amino acid substitutions that might affect the binding of the final immunogenicity-reduced molecule, a plurality of other variant V_{H} and V_{L} sequences are aslo designed.

The immunogenicity-reduced variable regions are constructed by the method of overlapping PCR recombination. The cloned non-human starting V_{H} and V_{L} genes are used as templates for mutagenesis of the framework regions to the required immunogenicity-reduced sequences. Sets of mutagenic primer pairs are synthesized encompassing the regions to be altered. In preferred embodiments, the vectors VH-PCR1 and VL-PCR1 (Riechmann et al., 1988) can be used as templates to introduce a 5′ flanking sequence, including the leader signal peptide, leader intron and the murine immunoglobulin promoter, and a 3′ flanking sequence, including the splice site and intron sequences. The immunogenicity-reduced V regions produced are then cloned into a suitable plasmid, e.g., pUC 19, and the entire DNA sequence is coiurmed to be correct for each immunogenicity-reduced V_{H} and V_{L}.

The immunogenicity-reduced heavy and light chain V-region genes can be excised from the plasmids as appropriate restriction fragments, which include the non-human heavy chain immunoglobulin promoter, the leader signal peptide, leader intron, the V_{H} or V_{L} sequence and the splice site. These are transferred to suitable expression vectors which include human constant regions, e.g., IgG1 constant regions, and markers for selection in mammalian cells.

The heavy and light chain expression vectors are preferably co-transfected in a variety of combinations into a suitable cell line by electroporation. Colonies expressing the selection marker gene are selected. Production of human antibody by transfected cell clones can be measured by ELISA for human IgG. Cell lines secreting antibody are selected and expanded. The immunogenicity-reduced antibodies are purified using standard method known in the art.

The immunogenicity-reduced antibodies are preferably screened for their binding affinities to RBCs. In a preferred embodiment, a modified antigen binding assay is used, in which the antibodies are reacted with RBCs in solution and the cells are then fixed to 96-well plates with poly L-lysine and glutaraldehyde at the end of the assay, just prior to the addition of the substrate. Washed erythrocytes are added to dilutions of antibody in 96-well V-bottom plates. Bound antibody is detected with biotinylated anti-human antibody or an antibody that binds the starting non-human antibody, then visualized using avidin alkaline phosphatase according to standard methods.

### 5.2 ANTIGEN-BINDING PORTION THAT BINDS A PATHOGENIC ANTIGENIC MOLECULE AND PRODUCTION

The present invention also provides immunogenicity-reduced bispecific molecules that comprise an immunogenicity-reduced anti-CR1 antibody as described in Section 5.1. and an antigen-binding portion which bind a pathogenic antigenic molecule.

Antibodies or antibody fragments against an antigen of interest (e.g., an antigen to be cleared from the circulation of a mammal) can be prepared by immunizing a suitable subject with an antigen as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see generally Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., 1975, Nature, 256:495, or may be made by recombinant DNA methods (U.S. Pat. No. 4,816,567). The term “monoclonal antibody” as used herein also indicates that the antibody is an immunoglobulin.

In the hybridoma method of generating monoclonal antibodies, a mouse or other appropriate host animal, such as a hamster, is immunized as hereinabove described to elicit lymphocytes that produce or are capable of producing antibodies that will specifically bind to the protein used for immunization (see generally, U.S. Pat. No. 5,914,112, which is incorporated herein by reference in its entirety.).

Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (RGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immuno-absorbent assay (ELISA). The binding affinity of the monoclonal antibody can, for example, be determined by the Scatchard analysis of Munson et al., 1980, Anal. Biochem., 107:220 or by surface plasmon resonance using, e.g., a Biacore instrument.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal antibody-secreting hybridomas, a monoclonal antibody directed against a pathogen or pathogenic antigenic molecule polypeptide of the invention can be identified and isolated by screening a recombinant combinatorial immunoglobulin library (e.g., an antibody phage display library) with the antigen of interest. Kits for generating and screening phage display libraries are commercially available (e.g., Pharmacia Recombinant Phage Antibody System, Catalog No. 27-9400-01; and the Stratagene antigen SurfZAP Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In addition, techniques developed for the production of “chimeric antibodies” (Morrison, et al., 1984, Proc. Natl. Acad. Sci., 81, 6851-6855; Neuberger, et al., 1984, Nature 312, 604-608; Takeda, et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.)

Humanized antibodies are antibody molecules from non-human species having one or more complementarity determining regions (CDRs) from the non-human species and a framework region from a human immunoglobulin molecule. (see e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Riechmann et al. (1988, Nature, 332:323; antibodies against hepatitis B in Cole et al. (1991, Proc. Natl. Acad. Sci. USA 88:2869); as well as against viral antigens-respiratory syncitial virus in Tempest et al. (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

Completely human antibodies are particularly desirable for therapeutic treatment of human patients. Such antibodies can be produced using transgenic mice which are incapable of expressing endogenous immunoglobulin heavy and light chain genes, but which can express human heavy and light chain genes. The transgenic mice are immunized in the normal fashion with a selected antigen, e.g., all or a portion of an immunogen.

Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see e.g., U.S. Pat. No. 5,625,126; U.S. Patent 5,633,425; U.S. Patent 5,569,825; U.S. Patent 5,661,016; and U.S. Patent 5,545,806. In addition, companies such as Abgenix, Inc. (Fremont, Calif., see, for example, U.S. Pat. No. 5,985,615) and Medarex, Inc. (Princeton, N.J.), can be engaged to provide human antibodies directed against a selected antigen using technology similar to that described above.

### 5.3 BISPECIFIC MOLECULES

The present invention provides immunogenicity-reduced bispecific molecules, e.g., immunogenicity-reduced bispecific antibodies, that are characterized by having an immunogenicity-reduced first antigen recognition portion that binds CR1 and a second antigen recognition portion that binds an epitope of an antigen of interest to be cleared from the circulation of a subject.

According to the invention, the first antigen recognition portion of a bispecific molecule can be any polypeptide that contains an immunogenicity-reduced anti-CR1 binding domain and an effector domain. In preferred embodiments, the immunogenicity-reduced anti-CR1 antibody comprises one or more non-human V_{H} or V_{L} sequences, in each of which one or more human T cell epitopes are modified by substitution of one or more amino acids. In preferred embodiments, the immunogenicity-reduced anti-CR1 antibodies comprising one or more of VH1-VH5 and one or more of VL1-VL2 as described in Section 5.1. The immunogenicity-reduced anti-CR1 binding portion can be any immunogenicity-reduced anti-CR1 molecules described in Section 5.1. In a preferred embodiment, the first antigen recognition portion is an immunogenicity-reduced anti-CR1 mAb. In a preferred embodiment, the immunogenicity-reduced anti-CR1 monoclonal antibody is 19E9,12H10, 15A12, 44H1, 31C11. In another embodiment, the first antigen recognition portion is an immunogenicity-reduced anti-CR1 polypeptide antibody, including but is not limited to, a single-chain variable region fragment (scFv) with specificity for a CR1 receptor fused to the N-terminus of an immunoglobulin Fc domain. The first antigen binding portion can also be an immunogenicity-reduced chimeric antibody, such as but is not limited to an immunogenicity-reduced humanized monoclonal antibody wherein the complementarity determining regions are mouse, and the framework regions are human thereby decreasing the likelihood of an immune response in human patients treated with the antibody (U.S. Pat. Nos. 4,816,567, 4,816,397, 5,693,762; 5,585,089; 5,565,332 and 5,821,337 which are incorporated herein by reference in their entirety). Preferably, the Fc domain of the chimeric antibody can be recognized by the Fc receptors on phagocytic cells, thereby facilitating the transfer and subsequent proteolysis of the RBC-immune complex. In a specific embodiment, the immunogenicity-reduced chimeric antibody is 3G4 which comprises E11 murine variable regions linked with human IgG1 constant regions.

According to the invention, the second antigen recognition portion of a bispecific molecule can be any molecular moiety, including but is not limited to any antibody or antigen binding fragment thereof, that recognizes and binds an antigen of interest. The antigenic molecule that the second antigen recognition portion binds can be any substance that is present in the circulation that is potentially injurious to or undesirable in the subject to be treated, including but is not limited to proteins or drugs or toxins, autoantibodies or autoantigens, or a molecule of any infectious agent or its products. An antigenic molecule is any molecule containing an antigenic determinant (or otherwise capable of being bound by a binding domain) that is or is part of a substance (e.g., a pathogen) that is the cause of a disease or disorder or any other undesirable condition.

The second antigen-binding recognition portion of the bispecific molecule of the invention can be an antibody, e.g., a monoclonal antibody, that recognizes and binds a pathogenic antigenic molecule. The antigen-binding portion of the bispecific molecule can also be any antigen binding fragment of an antibody which recognizes and binds an antigenic molecule. In another preferred embodiment, the antigen-binding antibody fragment is an Fab, an Fab′, an (Fab′)₂, or an Fv fragment of an immunoglobulin molecule. In another preferred embodiment, the antigen-binding antibody fragment is a single chain Fv (scFv) fragment which can be obtained, e.g., from a library of phage-displayed antibody fragments by affinity screening and subsequent recombinant expressing. In still another embodiment, the antigen-binding antibody fragment portion of the bispecific molecule is a single-chain antibody (scAb). As used herein, a single-chain antibody (scAb) includes antibody fragments consisting of an scFv fused with a constant domain, e.g., the constant κ domain, of a immunoglobulin molecule.

The second antigen recognition portion of the bispecific molecule can also be a non-proteinaceous moiety. In one embodiment, the second antigen recognition portion is a nucleic acid. In another embodiment, the second antigen recognition portion is an organic small molecule. In still another embodiment, the second antigen binding portion is an oligosaccharide.

Various purified bispecific molecules can be combined into a “cocktail” of bispecific molecules. As used herein, a cocktail of bispecific molecules of the invention refers to a mixture of purified bispecific molecules for targeting one or a mixture of antigens or pathogens. In particular, the cocktail of bispecific molecules refers to a mixture of purified bispecific molecules having a plurality of second antigen binding domains that target different or same anitigenic molecules and that are of mixed types. For example, the mixture of the second antigen binding domains can be a mixture of peptides, nucleic acids, and/or organic small molecules. A cocktail of bispecific molecules is generally prepared by mixing various purified bispecific molecules. Such bispecific molecule cocktails are useful, inter alia, as personalized medicine tailored according to the need of individual patients.

The bispecific molecule can be cross-linked antibodies, comprising an immunogenicity-reduced anti-CR1 antibody specific to a human CR1 receptor and a second antibody which is specific to a pathogenic antigenic molecule. The bispecific molecule can also be antibodies that are produced recombinantly and have an immunogenicity-reduced CR1 binding domain which recognizes a CR1 receptor and a second domain recognize a pathogenic antigenic molecule. The bispecific molecule can as well be produced using the method of protein trans-splicing and has a first antigen recognition portion which is an immunogenicity-reduced CR1 binding region and a second antigen recognizing portion recognizing a pathogenic antigenic molecule.

In one embodiment, the immunogenicity-reduced anti-CR1 bispecific molecule of the invention is a single molecule (preferably a polypeptide) which consists essentially of, or alternatively comprises, a first binding domain (BD1) bound to the amino terminus of a CH2 and CH3 portion of an immunoglobulin heavy chain (Fc) bound to a second binding domain (BD2) at the Fc domain's carboxy terminus. In another embodiment, the CH2 domain and the CH3 domain positions are present in reverse order. One of the binding domains binds CR1, and the other of the binding domains binds a pathogenic antigenic molecule. The binding domains can individually be a scFv (i.e., a V_{L} fused via a polypeptide linker to a V_{H}) or a receptor or ligand or binding domain thereof, or other binding partner, with the desired specificity. For example, the binding domain that binds the pathogenic antigenic molecule can be a cellular receptor for a virus (e.g., CD4 and/or a chemokine receptor, which bind to HIV), or a receptor for a bacteria (e.g., polymyxin or multimers thereof which bind to Gram-negative bacteria), or a cellular receptor for a drug or other molecule (e.g., ∀ domain of the IgE receptor which binds IgE, to treat or prevent allergic reactions), or a receptor for an autoantibody (e.g., acetylcholine receptor, for treating or preventing myasthenia gravis).

In an embodiment where a binding domain is not a polypeptide or is not otherwise readily expressed as a fusion protein with the other portions of the bispecific molecule, such binding domain can be cross-linked to the rest of the bispecific molecule. For example, polymyxin can be cross-linked to a fusion polypeptide comprising CH₂CH₃ and the binding domain that binds CR1.

In another embodiment, the bispecific molecule of the invention is a dimeric molecule consisting of a first molecule (preferably a polypeptide) consisting essentially of, or comprising, a BD1 bound to the amino terminus of an immunoglobulin Fc domain (a hinge region, a CH2 domain and a CH3 domain), and a second molecule (preferably a polypeptide), consisting essentially of, or comprising, a Fc domain with a BD2 domain bound to the Fc domain's carboxy terminus, wherein the Fc domains of the first and second polypeptides are complementary to and can associate with each other. BD1 and BD2 are as described above.

In a specific embodiment, one or both of the monomers of the bispecific molecule (preferably a polypeptide) consists essentially of, or comprises, a variable light chain domain (VL) and constant light chain domain (CL) followed by a linker molecule (of any structure/sequence) bound to the amino terminus of a variable heavy chain domain, followed by a CH1 domain, a hinge region, a CH2 domain, and a CH3 domain.

In a specific embodiment, one or both of the monomers of the bispecific molecule (preferably a polypeptide) consists essentially of, or comprises, a scFv bound to the amino terminus of a CH1 domain, followed by a hinge region, a CH2 domain and a CH3 domain.

In another embodiment, an immunogenicity-reduced anti-CR1 bispecific molecule of the invention is a molecule comprising two separate scFv with specificity for two separate antigens (one of which is CR1, the other of which is the pathogenic antigenic molecule). The molecule (preferably polypeptide) consists essentially of, or comprises, a first scFv domain bound to a CH2 domain, followed by a CH3 domain, and a second scFv domain.

In another embodiment, the bispecific molecule of the invention is a molecule consisting essentially of, or comprising, two variable regions with specificity for the two separate antigens. The molecule (preferably polypeptide) consists essentially of, or comprises, a first variable heavy chain domain bound to a variable light chain domain, followed by a CH2 domain, a CH3 domain, a variable heavy chain domain, and a variable light chain domain.

Alternatively, the positions of the CH2 and CH3 domains may be switched. Further, the invention contemplates that the domains may be further rearranged into different positions relative to one another, while retaining its functional properties, i.e., binding to CR1, binding to a pathogenic antigenic molecule, and capable of being cleared from the circulation by macrophages. Moreover, as will be clear from the discussion above, the binding domains described above preferably, but need not be, polypeptides (including peptides). Moreover, the binding domains can provide the desired binding specificity via covalent or noncovalent linkage to the appropriate structure that mediates binding. For example, the binding domain may contain avidin or streptavidin that is noncovalently bound to a biotinylated molecule that in turn binds a pathogen antigenic molecule.

Furthermore, the invention also encompasses immunogenicity-reduced bispecific molecules as prepared by the methods disclosed in WO 01/80883 and WO 02/46208, each of which is incorporated herein by reference in its entirety. For example, the position of two binding domains (BD1 and BD2) may be switched for the bispecific molecule.

### 5.3 METHOD OF MAKING BISPECIFIC MOLECULES: CHEMICAL CROSS-LINKING

The bispecific molecules used in the present invention can be produced by chemical cross-linking antibodies, see e.g., U.S. Pat. Nos. 5,487,890, 5,470,570, 5,879,679, PCT publication WO 02/075275, U.S. Provisional Application No. 60/411,731, filed on Sep. 16, 2002, U.S. Provisional Application No. 60/411,421, filed on Sep. 16, 2002, U.S. Provisional Application No. To be assigned, Attorney Docket No. 9635-046-888, filed on Mar. 28, 2003, each of which is incorporated herein by reference in its entirety.

In preferred embodiments of the invention, the bispecific molecule comprises an immunogenicity-reduced anti-CR1 mAb cross-linked to one or more antigen-binding antibody or antibody fragments. The anti-CR1 antibody, e.g., anti-CR1 mAb, and the antigen-binding antibody fragment(s) are preferably conjugated by cross-linking via a cross-linker. Any cross-linking chemistry known in art for conjugating proteins can be used in the conjunction with the present invention. In a preferred embodiment of the invention, the anti-CR1 mAb and the antigen-binding antibody fragment are produced using cross-linking agents sulfosuccinimidyl 4-(N-maleinzidomethyl) cyclohexane-1-carboxylate (sSMCC) and N-succinimidyl-S-acetyl-thioacetate (SATA). In another preferred embodiment of the invention, the anti-CR1 mAb and the antigen-binding antibody fragment are conjugated via a poly-(ethylene glycol) cross-linker (PEG). In this embodiment, the PEG moiety can have any desired length. For example, the PEG moiety can have a molecular weight in the range of 200 to 20,000 Daltons. Preferably, the PEG moiety has a molecular weight in the range of 500 to 1000 Daltons or in the range of 1000 to 8000 Daltons, more preferably in the range of 3250 to 5000 Daltons, and most preferably about 5000 Daltons. Such a bispecific molecule can be produced using cross-linking agents N-succinimidyl-S-acetyl-thioacetate (SATA) and a poly(ethylene glycol)-maleimide, e.g., monomethoxy poly(ethylene glycol)-maleimide (mPEG-MAL) or NHS-poly(ethylene glycol)-maleimide (PEG-MAL). Methods of producing PEG-linked bispecific molecules is described in U.S. Provisional Application No. 60/411,731, filed on Sep. 16, 2002.

### 5.3 METHOD OF MAKING BISPECIFIC MOLECULES: RECOMBINANT TECHNIQUES

The bispecific molecules used in the present invention can also be produced recombinantly, whereby nucleotide sequences that encode antibody variable domains with the desired binding specificities (antibody-antigen combining sites) are fused to nucleotide sequences that encode immunoglobulin constant domain sequences, see e.g., PCT publication WO 01/80883, which is incorporated herein by reference in its entirety. The fusion preferably is with an immunoglobulin heavy chain constant domain, comprising at least part of the hinge, CH2, and CH3 regions. It is preferred to also have the first heavy-chain constant region (CH1) containing an amino acid residue with a free thiol group so that a disulfide bond may be allowed to form during the translation of the protein in the hybridoma, between the variable domain and the heavy chain (see, Arathoon et al., WO 98/50431).

In a preferred embodiment of this approach, the bispecific antibodies are composed of a hybrid immunoglobulin heavy chain with a first binding specificity in one arm fused to the constant CH2 and CH3 domains, and a hybrid immunoglobulin heavy chain-light chain pair (providing a second binding specificity) in the other arm (see, e.g., WO 94/04690 published Mar. 3, 1994). In one embodiment, DNAs encoding the immunoglobulin heavy chain fusions and, if desired, the immunoglobulin light chain, are inserted into separate expression vectors, and are co-transfected into a suitable host organism. In another embodiment, the coding sequences for two or all three polypeptide chains are inserted in one expression vector. The bispecific molecules comprising single polypeptides can also be produced recombinantly. In one embodiment, the nucleic acid encoding an antigen recognition portion that binds a shed tumor antigen is fused to the nucleic acid encoding an antigen recognition portion that binds a CR1 receptor to obtain a fusion nucleic acids encoding a single polypeptide bispecific molecule. The nucleic acid is then expressed in a suitable host to produce the bispecific molecule.

In a specific embodiment, the bispecific molecule is produced by a method comprising producing a bispecific immunoglobulin-secreting cell that has a first antigen recognition portion that binds CR1 and a second antigen recognition portion that binds an epitope of a shed tumor associated antigen. The method comprises the steps of fusing a first cell expressing an immunoglobulin that binds to CR1 with a second cell expressing an immunoglobulin that binds to the shed tumor associated antigen, and selecting for cells that express the bispecific immunoglobulin. In another specific embodiment, a bispecific molecule comprising at least a first antigen recognition portion that binds CR1 and a second antigen recognition portion that binds an epitope of a shed tumor associated antigen is produced by a method comprising the steps of transforming a cell with a first DNA sequence encoding at least the first antigen recognition portion and a second DNA sequence encoding at least the second antigen recognition portion, and independently expressing said first DNA sequence and said second DNA sequence so that said first and second antigen recognition portions are produced as separate molecules that assemble together in said transformed single cell, that is capable of binding to CR1 with a first antigen recognition portion and also capable of binding an antigen to be cleared from the circulation with a second antigen recognition portion is formed.

### 5.3 METHOD OF MAKING BISPECIFIC MOLECULES: PROTEIN TRANS-SPLICING

The bispecific molecules used in the present invention can also be produced using the method of protein trans-splicing, see e.g., PCT publication WO 02/46208, which is incorporated herein by reference in its entirety. The method can be used to directly or via a linker conjugate a first antigen recognition portion, e.g., an anti-CR1 mAb, with a second antigen recognition portion that binds an epitope of a shed tumor associated antigen, e.g., a peptide or polypeptide, a nucleic acid, and an organic small molecules, to form a bispecific molecule. Alternatively, the method can be used to conjugate a first antigen recognition portion with streptavidin to form a first antigen recognition portion-streptavidin fusion molecule that can be conjugated with a biotinylated second antigen recognition portion.

In the method using protein trans-splicing, the first antigen recognition portion is conjugated to the N-terminus of an N-intein of a suitable split intein to produce an N-intein first antigen recognition portion fragment, whereas the second antigen recognition portion is conjugated to the C-terminus of the C-intein of the split intein to produce a C-intein second antigen recognition portion fragment. The N-intein first antigen recognition portion fragment and the C-intein second antigen recognition portion fragment are then brought together such that they reconstitute and undergo trans-splicing to produce the bispecific molecule.

The bispecific molecule produce by protein trans-splicing can contain a single second antigen recognition portion conjugated to the first antigen recognition portion. Alternatively, the bispecific molecule of the invention can also contain two or more second antigen recognition portions conjugated to different regions of the first antigen recognition portion. For example, the bispecific molecule can contain two second antigen recognition portions conjugated to each of the heavy chains of a first antigen recognition monoclonal antibody. When two or more second antigen recognition portions are contained in the bispecific molecule, such second antigen recognition portions can be the same or different antigen recognition portions. The first and second antigen recognition portions can be different antigen recognition portions that target the same shed tumor associated antigen to be cleared. In a preferred embodiment of the invention, the first and second antigen recognition portions target an antigenic molecule to be cleared cooperatively. As a non-limiting example, one of the second antigen recognition portions may enhance the binding of the other second antigen recognition portion to a shed tumor associated antigen, thereby facilitating the removal of the shed tumor associated antigen. The first and second antigen recognition portions can also be different antigen recognition portions that target different shed tumor associated antigens to be cleared.

Various split inteins can be used for the production of the bispecific molecules of the invention. In one aspect of the invention, naturally occurring split inteins are used for the production of bispecific molecules. In another aspect of the invention, engineered split intein based on naturally occurring non-split inteins are used for the production of bispecific molecules. In various embodiments of the invention, a split intein can be modified by adding, deleting, and/or mutating one or more amino acid residues to the N-intein and/or the C-intein such that the modification improves or enhances the intein's proficiency in trans-splicing and/or permits control of trans-splicing processes. In one preferred embodiment, a Cys residue can be included at the carboxy terminus of a C-intein so that the requirement that the molecular moiety conjugated to the C-intein must start with a Cys is alleviated. In other preferred embodiments, one or more native proximal extein residues are added to the −and/or C-intein to facilitate trans-splicing in a foreign extein content.

In a preferred embodiment, the trans-splicing system of the split intein encoded in the DnaE gene of Synechocystis sp. PCC6803 is used for the production of the bispecific molecules of the invention. In another embodiment of the invention, an engineered split intein system based on the Mycobacterium tuberculosis RecA intein is used. The production of bispecific molecules can be carried out in vitro wherein the intein antigen recognition portion fragments are expressed in separate hosts. The production of bispecific molecules can also be carried out in vivo. In one embodiment, nucleic acids encoding the intein antigen recognition portion fragments are inserted into separate vectors, which are then co-transfected into a host for in vivo production of the bispecific molecule. In another embodiment, nucleic acids encoding the intein fragments are inserted into the same vector, which is then transfected into a host for in vivo production of the bispecific molecule.

In the method, the N-intein first antigen recognition portion fragment is preferably produced by fusing an appropriate antigen recognition moiety that binds CR1 to the N-terminus of the N-intein of a suitable split intein. In a preferred embodiment, the C-terminus of the heavy chain of an anti-CR1 mAb is fused to the N-terminus of the N-intein of a split intein. The C-intein second antigen recognition portion fragment is preferably produced by fusing an appropriate antigen recognition moiety that binds an epitope of a shed tumor associated antigen to be cleared to the C-terminus of the C-intein of a suitable split intein. The amino acid residue immediately at the C-terminal side of the splice junction of the C-intein is a cysteine, serine, or threonine. In another embodiment of the invention, a C-intein streptavidin is produced by fusing a streptavidin to the C-terminus of a C-intein comprising a Cys, Ser, or Thr immediately downstream of the splice junction and is used in trans-splicing to produce a first antigen recognition portion-streptavidin fusion molecule, which subsequently reacts with a biotinylated second antigen recognition portion to produce the bispecific molecule. It is also understood that other molecules that specifically bind biotin, including but not limited to avidin, are also within the scope of the invention.

In one embodiment, the bispecific molecule is produced by mixing the N-intein first antigen recognition portion fragment and the C-intein second antigen recognition portion fragment in vitro so that the fragments reconstitute and undergo trans-splicing. In another embodiment, a first antigen recognition portion-streptavidin molecule is produced by mixing the N-intein first antigen recognition portion fragment and the C-intein streptavidin fragment in vitro to produce a first antigen recognition portion-streptavidin molecule. The bispecific molecule is then produced by reaction of the first antigen recognition-streptavidin molecule with a biotinylated second antigen recognition portion.

### 5.3 EX VIVO PREPARATION OF THE BISPECIFIC MOLECULE

In an alternative embodiment, the bispecific molecule, such as a bispecific antibody, is prebound to hematopoietic cells of the subject ex vivo, prior to administration. For example, hematopoietic cells are collected from the individual to be treated (or alternatively hematopoietic cells from a non-autologous donor of the compatible blood type are collected) and incubated with an appropriate dose of the therapeutic bispecific antibody for a sufficient time so as to allow the antibody to bind CR1 on the surface of the hematopoietic cells. The hematopoietic cell/bispecific antibody mixture is then administered to the subject to be treated in an appropriate dose (see, for example, Taylor et al., U.S. Pat. No. 5,487,890).

The hematopoietic cells are preferably blood cells, most preferably red blood cells.

Accordingly, in a specific embodiment, the invention provides a method of treating a mammal having an undesirable condition associated with the presence of a pathogenic antigenic molecule, comprising the step of administering a hematopoietic cell/bispecific molecule complex to the subject in a therapeutically effective amount, said complex consisting essentially of a hematopoietic cell expressing CR1 bound to one or more bispecific molecules, wherein said bispecific molecule (a) does not consist of a first monoclonal antibody to CR1 that has been chemically cross-linked to a second monoclonal antibody, (b) comprises a first binding domain which binds CR1 on the hematopoietic cell, and (c) comprises a second binding domain which binds the pathogenic antigenic molecule. The method alternatively comprises a method of treating a mammal having an undesirable condition associated with the presence of a pathogenic antigenic molecule comprising the steps of (a) contacting a bispecific molecule with hematopoietic cells expressing CR1, to form a hematopoietic cell/bispecific molecule complex, wherein the bispecific molecule (i) does not consist of a first monoclonal antibody to CR1 that has been chemically cross-linked to a second monoclonal antibody, (ii) comprises a first binding domain which binds CR1, and (iii) comprises a second binding domain which binds the pathogenic antigenic molecule; and (b) administering the hematopoietic cell/bispecific molecule complex to the mammal in a therapeutically effective amount.

The invention also provides a method of making a hematopoietic cell/bispecific molecule complex comprising contacting a bispecific molecule with hematopoietic cells that express CR1 under conditions conducive to binding, such that a complex forms, said complex consisting essentially of a hematopoietic cell bound to one or more bispecific molecules, wherein said bispecific molecule (a) comprises a first binding domain that binds CR1 on the hematopoietic cells, (b) comprises a second binding domain that binds a pathogenic antigenic molecule, and (c) does not consist of a first monoclonal antibody to CR1 that has been chemically cross-linked to a second monoclonal antibody.

Taylor et al. (U.S. Pat. No. 5,879,679, hereinafter “the '679 patent”) have demonstrated in some instances that the system saturates because the concentration of autoantibodies (or other pathogenic antigen) in the plasma is so high that even at the optimum input of bispecific antibodies, not all of the autoantibodies can be bound to the hematopoietic cells under standard conditions. For example, for a very high titer of autoantibody sera, a fraction of the autoantibody is not bound to the hematopoietic cells due to its high concentration.

However, saturation can be solved by using combinations of bispecific antibodies which contain monoclonal antibodies that bind to different sites on CR1. For example, the monoclonal antibodies 19E9 and 12H10 bind to separate and non-competing sites on the primate C3b receptor. Therefore, a “cocktail” containing a mixture of two bispecific antibodies, each made with a different monoclonal antibody to CR1, may give rise to greater binding of antibodies to red blood cells. The bispecific antibodies of the invention can also be used in combination with certain fluids used for intravenous infusions.

In yet another embodiment, the bispecific molecule, such as a bispecific antibody, is prebound to red blood cells in vitro as described above, using a “cocktail” of at least two different bispecific antibodies. In this embodiment, the two different bispecific antibodies bind to the same antigen, but also bind to distinct and non-overlapping recognition sites on CR1. By using at least two non-overlapping bispecific antibodies for binding to CR1, the number of bispecific antibody-antigen complexes that can bind to a single red blood cell is increased. Thus, by allowing more than one bispecific antibody to bind to a single CR1, antigen clearance is enhanced, particularly in cases where the antigen is in very high concentrations (see for example the '679 patent, column 6, lines 41-64).

### 5.3 POLYCLONAL POPULATIONS OF BISPECIFIC MOLECULES

The invention also provides polyclonal population of immunogenicity-reduced bispecific molecules. As used herein, a polyclonal population of immunogenicity-reduced bispecific molecules of the present invention refers to a population of bispecific molecules, comprising a plurality of different immunogenicity-reduced bispecific molecules each having a first antigen recognition region that binds a pathogenic antigenic molecule and a second antigen recognition region that binds CR1, wherein the first antigen recognition regions in the plurality of different bispecific molecules are each different and each have a different binding specificity and wherein each of said bispecific molecules does not consist of a first monoclonal antibody that has been chemically cross-linked to a second monoclonal antibody to CR1. In some embodiments, the first and second antigen recognition regions of each bispecific molecule in the polyclonal population do not comprise more than one heavy and light chain pair. Preferably, the plurality of bispecific molecules of the polyclonal population includes specificities for different epitopes of an antigenic molecule and/or for different variants of an antigenic molecule. More preferably, the plurality of bispecific molecules of the polyclonal population includes specificities for the majority of naturally-occurring epitopes of an antigenic molecule and/or for all variants of an antigenic molecule. The polyclonal population can also include specificities for a mixture of different antigenic molecules. In preferred embodiments, at least 90%, 75%, 50%, 20%, 10%, 5%, or 1% of bispecific molecules in the polyclonal population target the desired antigenic molecule and/or antigenic molecules. In other preferred embodiments, the proportion of any single bispecific molecule in the polyclonal population does not exceed 90%, 50%, or 10% of the population. The polyclonal population comprises at least 2 different bispecific molecules with different specificities. More preferably, the polyclonal population comprises at least 10 different bispecific molecules with different specificities. Most preferably, the polyclonal population comprises at least 100 different bispecific molecules with different specificities.

The polyclonal populations of bispecific molecules of the invention can be used for more efficient clearance of pathogens that have multiple epitopes and/or pathogens that have multiple variants or mutants, which normally cannot be effectively targeted and cleared by a monoclonal antibody having a single specificity. By targeting multiple epitopes and/or multiple variants of a pathogen, the polyclonal population of bispecific molecules is advantageous in the clearance of pathogens that have a higher mutation rate because simultaneous mutations at more than one epitopes tend to be much less frequent.

The polyclonal populations of bispecific molecule of the invention can comprise any type of bispecific molecules described previously in Section 5.3. The polyclonal populations of bispecific molecules are produced by adapting any methods described in Sections 5.3.1 through 5.3.3.

The polyclonal population of bispecific molecules of the invention can be produced by transfecting a hybridoma cell line that expresses immunogenicity-reduced immunoglobulin that binds CR1 with a population of eukaryotic expression vectors containing nucleic acids encoding the heavy and light chain variable regions of a polyclonal population of immunoglobulins that bind different antigenic molecules. Cells that express bispecific immunoglobulins that comprise a first binding domain which binds to a pathogenic antigenic molecule and a second binding domain which binds to CR1 are then selected using standard methods known in the art. The polyclonal population of immunoglobulins can be obtained by any method known in the art, e.g., from a phage display library. If a phage display library is used, the number of specificities of such phage display library is preferably near the number of different specificities that are expressed at any one time by lymphocytes. More preferably the number of specificities of the phage display library is higher than the number of different specificities that are expressed at any one time by lymphocytes. Most preferably the phage display library comprises the complete set of specificities that can be expressed by lymphocytes. Kits for generating and screening phage display libraries are commercially available (e.g., Pharmacia Recombinant Phage Antibody System, Catalog No. 27-9400-01; and the Stratagene antigen SurfZAP Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In a preferred embodiment, the polyclonal population of eukaryotic expression vectors is produced from a phage display library according to Den et al., 1999, J. Immunol. Meth. 222:45-57. The phage display library is screened to select a polyclonal sublibrary having binding specificities directed to the antigenic molecule or antigenic molecules of interests by affinity chromatography (McCafferty et al., 1990, Nature 248:552; Breitling et al., 1991, Gene 104:147; and Hawkins et al., 1992, J. Mol. Biol. 226:889). The nucleic acids encoding the heavy and light chain variable regions are then linked head to head to generate a library of bidirectional phage display vectors. The bidirectional phage display vectors are then transferred in mass to bidirectional mammalian expression vectors (Sarantopoulos et al., 1994, J. Immunol. 152:5344) which are used to transfect the hybridoma cell line.

In other preferred embodiments, the polyclonal population of bispecific molecules is produced by a method using the whole collection of selected displayed antibodies without clonal isolation of individual members as described in U.S. Pat. No. 6,057,098, which is incorporated by reference herein in its entirety. Polyclonal antibodies are obtained by affinity screening of a phage display library having a sufficiently large repertoire of specificities with an antigenic molecule having multiple epitopes, preferably after enrichment of displayed library members that display multiple antibodies. The nucleic acids encoding the selected display antibodies are excised and amplified using suitable PCR primers. The nucleic acids can be purified by gel electrophoresis such that the full length nucleic acids are isolated. Each of the nucleic acids is then inserted into a suitable expression vector such that a population of expression vectors having different inserts is obtained. In one embodiment, the population of expression vectors is then co-expressed with vectors containing a nucleotide sequence encoding an anti-CR1 binding domain in a suitable host. In another embodiment, the population of expression vectors and the vectors containing a nucleotide sequence encoding an anti-CR1 binding domain are expressed in separate hosts and the antigen binding domains and the anti-CR1 binding domain are combined in vitro to form the polyclonal population of bispecific molecules.

In still other embodiments, the polyclonal populations of bispecific antibodies are produced recombinantly, whereby the polyclonal population of nucleic acids which encode antibody variable domains with the desired binding specificities (antibody-antigen combining sites) are fused to nucleotides which encode immunoglobulin constant domain sequences. The fusion preferably is with an immunoglobulin heavy chain constant domain, comprising at least part of the hinge, CH2, and CH3 regions. It is preferred to also have the first heavy-chain constant region (CH1) containing an amino acid residue with a free thiol group so that a disulfide bond may be allowed to form during the translation of the protein in the hybridoma, between the variable domain and heavy chain (see, Arathoon et al., WO 98/50431).

DNAs encoding the immunoglobulin heavy chain fusions and, if desired, the immunoglobulin light chain, are inserted into separate expression vectors, and are co-transfected into a suitable-host organism. This provides for the ability to adjust the proportions of each of the three polypeptide fragments in unequal ratios of the three polypeptide chains, thus providing optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, each bispecific molecule in the polyclonal population is composed of a hybrid immunoglobulin heavy chain with a different first binding specificity in one arm fused to the constant CH2 and CH3 domains, and a hybrid immunoglobulin heavy chain-light chain pair (providing a second binding specificity) in the other arm. It was found that this asymmetric structure facilitates the separation of the desired bispecific compounds from unwanted immunoglobulin chain combinations, as the presence of an immunoglobulin light chain in only one half of the bispecific molecule provides for a facile way of separation. This approach is disclosed in WO 94/04690 published Mar. 3,1994.

Polyclonal populations of bispecific molecules comprising single polypeptide bispecific molecules can be produced recombinantly. A polyclonal population of nucleic acids encoding a polyclonal population of selected antigen recognition regions is fused to nucleic acids encoding the antigen recognition region that binds CR1 to obtain a population of fusion nucleic acids encoding a population of bispecific molecules. The population of nucleic acids are then expressed in a suitable host to produce a polyclonal population of bispecific molecules. In a preferred embodiment, the polyclonal population of nucleic acids encoding a polyclonal library of selected antigen recognition regions are obtained according to the method described in U.S. Pat. No. 6,057,098.

In still other preferred embodiments, the polyclonal population of bispecific molecules is produced from a population of displayed antibodies obtained by affinity screening with a set of antigens, such as but are not limited to a set of variants of a pathogen and/or a mixture of various pathogens. Such polyclonal population of bispecific molecules can be used to target and clear a set of antigens.

The polyclonal populations of bispecific molecules can be purified using any methods known in the art. The content of a polyclonal population of bispecific molecules can be determined using standard methods known in the art.

Although polyclonal populations of bispecific molecules produced from phage display libraries are described, it will be recognized by one skilled in the art that the plurality of second antigen recognition portions used in the generation of a population can be obtained from any population of suitable antigen recognition moieties. Populations of bispecific molecules produced from such population of antigen recognition moieties are intended to be within the scope of the invention.

### 5.3 COCKTAILS OF BISPECIFIC MOLECULES

Various purified bispecific molecules can be combined into a “cocktail” of bispecific molecules. As used herein, a cocktail of bispecific molecules of the invention refers to a mixture of purified bispecific molecules for targeting one or a mixture of antigens. In particular, the cocktail of bispecific molecules refers to a mixture of purified bispecific molecules having a plurality of first antigen binding domains that target different or same antigenic molecules and that are of mixed types. For example, the mixture of the first antigen binding domains can be a mixture of peptides, nucleic acids, and/or organic small molecules. A cocktail of bispecific molecules is generally prepared by mixing various purified bispecific molecules. Such bispecific molecule cocktails are useful, inter alia, as personalized medicine tailored according to the need of individual patients.

### 5.4 TARGET PATHOGENIC ANTIGENIC MOLECULES

The present invention provides methods of treating or preventing a disease or disorder associated with the presence of a pathogenic antigenic molecule. The pathogenic antigenic molecule can be any substance that is present in the circulation that is potentially injurious to or undesirable in the subject to be treated, including but not limited to an antigen of a pathogen, an autoantigen or a blood-borne protein desired to be removed from the circulatory system of a mammal. A pathogenic antigenic molecule is any molecule containing an antigenic determinant (or otherwise capable of being bound by a binding domain) that is or is part of a substance (e.g., a pathogen) that is the cause of a disease or disorder or any other undesirable condition.

Circulating pathogenic antigenic molecules cleared by the fixed tissue phagocytes include any antigenic moiety that is harmful to the subject. Examples of harmful pathogenic antigenic molecules include any pathogenic antigen associated with a parasite, fungus, protozoa, bacteria, or virus. Furthermore, circulating pathogenic antigenic molecules may also include toxins, e.g., anthrax protective antigen and lethal factor, botulinum, snake venom, etc.; immune complexes; autoantibodies; drugs; an overdose of a substance, such as a barbiturate; or anything that is present in the circulation and is undesirable or detrimental to the health of the host mammal. Failure of the immune system to effectively remove the pathogenic antigenic molecules from the mammalian circulation can lead to traumatic and hypovolemic shock (Altura and Hershey, 1968, Am. J. Physiol. 215:1414-9).

Moreover, non-pathogenic antigens, for example transplantation antigens, are mistakenly perceived to be harmful to the host and are attacked by the host immune system as if they were pathogenic antigenic molecules. The invention further provides an embodiment for treating transplantation rejection comprising administering to a subject an effective amount of a bispecific antibody that will bind and remove immune cells or factors involved in transplantation rejection, e.g., transplantation antigen specific antibodies.

### 5.4 AUTOIMMUNE ANTIGENS

In one embodiment, the pathogenic antigenic molecule to be cleared from the circulation includes autoimmune antigens. These antigens include but are not limited to autoantibodies or naturally occurring molecules associated with autoimmune diseases.

Many different autoantibodies can be cleared from the circulation of a primate by using the bispecific antibodies of the invention. In a non-limiting example, IgE (immunoglobulin E) antibodies are cleared from the circulation by the bispecific antibodies of the invention. More specifically, the bispecific antibodies comprise one variable region that is specific to an IgE and a second variable region that is specific to CR1. This bispecific antibody can be used to decrease circulating IgE antibodies thereby reducing or inhibiting allergic reactions such as asthma.

In another example, certain humans with hemophilia have been shown to be deficient in factor VIII. Recombinant factor VIII replacement treats this hemophilia. However, eventually some patients develop antibodies against factor VIII, thus interfering with the therapy. The bispecific antibodies of the invention prepared with an anti-anti-factor VIII antibodies provides a therapeutic solution for this problem. In particular, a bispecific antibody with specificity of the first variable region to anti-factor VIII autoantibodies and specificity of the second variable region to CR1 would be therapeutically useful in clearing the autoantibodies from the circulation, thus, ameliorating the disease.

Further examples of autoantibodies which can be cleared by the bispecific antibodies of the invention include, but are not limited to, autoantibodies to the following antigens: the muscle acetylcholine receptor (the antibodies are associated with the disease myasthenia gravis); cardiolipin (associated with the disease lupus); platelet associated proteins (associated with the disease idiopathic thrombocytopenic purpurea); the multiple antigens associated with Sjogren's Syndrome; the antigens implicated in the case of tissue transplantation autoimmune reactions; the antigens found on heart muscle (associated with the disease autoimmune myocarditis); the antigens associated with immune complex mediated kidney disease; the dsDNA and ssDNA antigens (associated with lupus nephritis); desmogleins and desmoplakins (associated with pemphigus and pemphigoid); or any other antigen which is characterized and is associated with disease pathogenesis.

When the above bispecific antibodies are injected into the circulation of a human or non-human primate, the bispecific antibodies will bind to red blood cells via the human or primate C3b receptor variable domain recognition site, at a high percentage and in agreement with the number of CR1 sites on red blood cells. The bispecific antibodies will simultaneously associate with the autoantibody indirectly, through the antigen, which is bound to the monoclonal antibody. The red blood cells which have the bispecific antibody/autoantibody complex on their surface then facilitate the removal and clearance from the circulation of the bound pathogenic autoantibody.

According to the invention, the bispecific antibodies facilitate pathogenic antigen or autoantibody binding to hematopoietic cells expressing CR1 on their surface and subsequently clear the pathogenic antigen or autoantibody from the circulation, without also clearing the hematopoietic cells.

### 5.4 INFECTIOUS DISEASES

In specific embodiments, infectious diseases are treated or prevented by administration of a bispecific molecule that binds both an antigen of an infectious disease agent and CR1. Thus, in such an embodiment, the pathogenic antigenic molecule is an antigen of an infectious disease agent.

Such antigen can be but is not limited to: influenza virus hemagglutinin (Genbank accession no. JO2132; Air, 1981, Proc. Natl. Acad. Sci. USA 78:7639-7643; Newton et al., 1983, Virology 128:495-501), human respiratory syncytial virus G glycoprotein (Genbank accession no. Z33429; Garcia et al., 1994, J. Virol.; Collins et al., 1984, Proc. Natl. Acad. Sci. USA 81:7683), envelop protein, matrix protein or other protein of Dengue virus (Genbank accession no. M19197; Hahn et al., 1988, Virology 162:167-180), measles virus hemagglutinin (Genbank accession no. M81899; Rota et al., 1992, Virology 188:135-142), herpes simplex virus type 2 glycoprotein gB (Genbank accession no. M14923; Bzik et al., 1986, Virology 155:322-333), poliovirus I VP1 (Emini et al., 1983, Nature 304:699), envelope glycoproteins of HIV I (Putney et al., 1986, Science 234:1392-1395), hepatitis B surface antigen (Itoh et al., 1986, Nature 308:19; Neurath et al., 1986, Vaccine 4:34), diphtheria toxin (Audibert et al., 1981, Nature 289:543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin-neuraminidase, swine flu hemagglutinin, swine flu neuraminidase, foot and mouth disease virus, hog colera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I), a glycoprotein of La Crosse virus (Gonzales-Scarano et al., 1982, Virology 120:42), neonatal calf diarrhea virus (Natsuno and Inouye, 1983, Infection and Immunity 39:155), Venezuelan equine encephalomyelitis virus (Mathews and Roehrig, 1982, J. Immunol. 129:2763), punta toro virus (Dalrymple et al., 1981, Replication of Negative Strand Viruses, Bishop and Compans (eds.), Elsevier, N.Y., p. 167), murine leukemia virus (Steeves et al., 1974, J. Virol. 14:187), mouse mammary tumor virus (Massey and Schochetman, 1981, Virology 115:20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495), of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase, bovine viral diarrhea virus glycoprotein 48 or glycoprotein 53.

Additional diseases or disorders that can be treated or prevented by the use of a bispecific molecule of the invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsachie virus, mumps virus, measles virus, rubella virus, polio virus, human immunodeficiency virus type I (HIV-I), and human immunodeficiency virus type II (HIV-II), any picornaviridae, enteroviruses, caliciviridae, any of the Norwalk group of viruses, togaviruses, such as Dengue virus, alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T-cell leukemia virus type I, human T-cell leukemia virus type II, simian immunodeficiency virus, lentiviruses, polyomaviruses, parvoviruses, Epstein-Barr virus, human herpesvirus-6, cercopithecine herpes virus 1 (B virus), and poxviruses

Bacterial diseases or disorders that can be treated or prevented by the use of bispecific molecules of the invention include, but are not limited to, Mycobacteria, Rickettsia, Mycoplasma, Neisseria spp. (e.g., Neisseria meningitides and Neisseria gonorrhoeae), Legionella, Vibrio cholerae, Streptococci, such as Streptococcus pneumoniae, Staphylococcus aureus, Staphylococcus epidermidis, Pseudomonas aeruginosa, Corynobacteria diptheriae, Clostridium spp., enterotoxigenic Eschericia coli, and Bacillus anthracis (anthrax), etc.

Protozoal diseases or disorders that can be treated or prevented by the use of bispecific molecules of the invention include, but are not limited to, plasmodia, eimeria, Leishmania, and trypanosoma.

### 5.4 ADDITIONAL PATHOGENIC ANTIGENIC MOLECULES

In one embodiment, the pathogenic antigenic molecule to be cleared from the circulation by the methods and compositions of the present invention encompass any serum drug, including but not limited to barbiturates, tricyclic antidepressants, and Digitalis.

In another embodiment, the pathogenic antigenic molecule to be cleared includes any serum antigen that is present as an overdose and can result in temporary or permanent impairment or harm to the subject. This embodiment particularly relates to drug overdoses.

In another embodiment, the pathogenic antigenic molecule to be cleared from the circulation include naturally occurring substances. Examples of naturally occurring pathogenic antigenic molecules that could be removed by the methods and compositions of the invention include but are not limited to low density lipoproteins, interleukins or other immune modulating chemicals and hormones.

### 5.5 DOSE OF BISPECIFIC ANTIBODIES

The dosage of immunogenicity-reduced bispecific molecules can be determined by routine experiments that are familiar to one skilled in the art. It can be determined based on the antigen level in the circulation, the half life of the bispecific molecule, as well as the number of RBCs and the number of CR1 sites on each RBC. The antigen level in the circulation can be determined by any technology known in the art, e.g., ELISA. The half life of the immunogenicity-reduced bispecific molecule can also be determined by different experiments, e.g., using ELISA to measure serum concentration of the bispecific molecules at different time points. The half life of an immunogenicity-reduced bispecific molecule depends both on the bispecific molecule itself and the particular antigen and amount of antigen the bispecific molecule complexes to.

The effects or benefits of administration of immunogenicity-reduced bispecific molecules can be evaluated by any methods known in the art, e.g., by methods that based on measuring the survival rate, side effects, clearance rate of the antigen of interest, or any combinations thereof. If the administration of an immunogenicity-reduced bispecific molecule achieves any one or more of the benefits in a patient, such as increasing the survival rate, decreasing side effects, increasing the clearance rate of an antigen of interest, the method is said to have efficacy.

The dose can be determined by a physician upon conducting routine experiments. Prior to administration to humans, the efficacy is preferably shown in animal models, e.g., primates or any animal model expressing primate or human CR1. Any animal model for a circulatory disease known in the art can be used.

More particularly, the dose of the bispecific antibody can be determiined based on the hematopoietic cell concentration and the number of CR1 epitope sites bound by the anti-CR1 receptor monoclonal antibodies per heinatopoietic cell. If the bispecific antibody is added in excess, a fraction of the bispecific antibody will not bind to hematopoietic cells, and will inhibit the binding of pathogenic antigens to the hematopoietic cell. The reason is that when the free bispecific antibody is in solution, it will compete for available pathogenic antigen with bispecific antibody bound to hematopoietic cells. Thus, the bispecific antibody-mediated binding of the pathogenic antigens to hematopoietic cells follows a bell-shaped curve when binding is examined as a function of the concentration of the input bispecific antibody concentration.

Viremia may result in up to 10⁸-10⁹ viral particles/ml of blood (HIV is 10⁶/ml; see, Ho, 1997, J. Clin. Invest. 99:2565-2567); the dose of therapeutic bispecific antibodies should preferably be, at a minimum, approximately 10 times the antigen number in the blood.

In general, for antibodies, the preferred dosage is 0.01 mg/kg to 10 mg/kg of body weight (generally 0.1 mg/kg to 5 mg/kg). Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et al., 1997, J. Acquired Immune Deficiency Syndromes and Human Retrovirology 14:193.

As defined herein, a therapeutically effective amount of bispecific antibody (i.e., an effective dosage) ranges from about 0.001 to 10 mg/kg body weight, preferably about 0.01 to 5 mg/kg body weight, more preferably about 0.1 to 2 mg/kg body weight, and even more preferably about 0.1 to 1 mg/kg, 0.2 to 1 mg/kg, 0.3 to 1 mg/kg, 0.4 to 1 mg/kg, or 0.5 to 1 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a bispecific antibody can include a single treatment or, preferably, can include a series of treatments. In a preferred example, a subject is treated with a bispecific antibody in the range of between about 0.1 to 5 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a bispecific antibody, used for treatment may increase or decrease over the course of a particular treatment. Changes in dosage may result and become apparent from the results of diagnostic assays as described herein.

It is understood that appropriate doses of bispecific antibody agents depends upon a number of factors within the ken of the ordinarily skilled physician, veterinarian, or researcher. The dose(s) of the bispecific antibody will vary, for example, depending upon the identity, size, and condition of the subject or sample being treated, further depending upon the route by which the composition is to be administered, if applicable, and the effect which the practitioner desires the bispecific antibody to have upon a pathogenic antigenic molecule or autoantibody.

It is also understood that appropriate doses of bispecific antibodies depend upon the potency of the bispecific antibody with respect to the antigen to be cleared. Such appropriate doses may be determined using the assays described herein. When one or more of these bispecific antibodies is to be administered to an animal (e.g., a human) in order to clear an antigen, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the RBC CR1 number, the activity of the bispecific antibody employed, the age, body weight, general health, gender, and diet of the subject, the time of administration, the route of administration, the rate of excretion, any drug combination, and the concentration of antigen to be cleared.

### 5.6 PHARMACEUTICAL FORMULATION AND ADMINISTRATION

The bispecific antibodies of the invention can be incorporated into pharmaceutical compositions suitable for administration. Such compositions typically comprise bispecific antibody and a pharmaceutically acceptable carrier. As used herein the language “pharmaceutically acceptable carrier” is intended to include any and all solvents, dispersion media, coatings, antibacterial and antifingal agents, isotonic and absorption delaying agents, and the like, compatible with pharmaceutical administration. The use of such media and agents for pharmaceutically active substances is well known in the art. Except insofar as any conventional media or agent is incompatible with the bispecific antibody, use thereof in the compositions is contemplated. Supplementary bispecific antibodies can also be incorporated into the compositions.

A pharmaceutical composition of the invention is formulated to be compatible with its intended route of administration. The preferred route of administration is intravenous. Other examples of routes of administration include parenteral, intradermal, subcutaneous, transdermal (topical), and transmucosal. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that the viscosity is low and the bispecific antibody is injectable. It must be stable under the conditions of manufacture and storage and must be preserved against the contaminating action of microorganisms such as bacteria and fungi.

The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the bispecific antibody (e.g., one or more bispecific antibodies) in the required amount in an appropriate solvent with one or a combination of ingredients enumerated above, as required, followed by filtered sterilization. Generally, dispersions are prepared by incorporating the bispecific antibody into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In one embodiment, the bispecific antibodies are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated herein by reference in its entirety.

It is advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of bispecific antibody calculated to produce the desired therapeutic effect in association with the required pharmaceutical carrier. The specification for the dosage unit forms of the invention are dictated by and directly dependent on the unique characteristics of the bispecific antibody and the particular therapeutic effect to be achieved, and the limitations inherent in the art of compounding such a bispecific antibody for the treatment of individuals.

The pharmaceutical compositions can be included in a kit, in a container, pack, or dispenser together with instructions for administration.

### 5.7 COMBINATION OF THERAPIES

It will be apparent to one skilled in the art that any of the therapies using bispecific molecules as described herein can be combined to maximize efficacy in treatment of diseases in a patient. Anyone skilled in the art will be able to determine the optimal combination of therapies for individual patient.

### 5.8 KITS

The invention also provides kits containing the immunogenicity-reduced bispecific molecules of the invention, or one or more nucleic acids encoding polypeptide immunogenicity-reduced bispecific molecules of the invention, and/or cells transformed with such nucleic acids, in one or more containers. The nucleic acids can be integrated into the chromosome, or exist as vectors (e.g., plasmids, particularly plasmid expression vectors). Kits containing the pharmaceutical compositions of the invention are also provided.

### 6. EXAMPLES

### Immunogenicity-Reduced Anti-CR1 Antibody and Bispecific Molecule Comprising Immunogenicity-Reduced Anti-CR1 Antibody

The following examples are presented by way of illustration of the present invention, and are not intended to limit the present invention in any way.

### 6.1 Example 1

### Immunogenicity-Reduced Antibody Against the Human Erythrocyte Complement Receptor 1 (CR1)

This example discloses the development of immunogenicity-reduced antibodies against the human erythrocyte complement receptor 1 (CR1).

Determination of Sequence of Murine Antibody Genes

The murine hybridoma E11 (Catalog# 184-020, Ancell Immunology Research Products MN) was propagated from a growing stock of cells in Dulbecco's Modified Eagle's medium supplemented with 10% fetal calf serum. The isotype of the antibody secreted was confirmed as mouse IgG16.

Total RNA was prepared from 10⁷ hybridoma cells. The conditioned medium from these cells was tested by ELISA for mouse antibody production, which was confirmed.

V_{H} and V_{L} cDNA was prepared using mouse 6 constant region and mouse IgG constant region primers. The first strand cDNAs were amplified by PCR using a variety of mouse signal sequence primers (six sets for V_{H} and seven sets for V_{L}. The amplified DNAs were gel-purified and cloned into the vector pGem® T Easy (Promega) according to standard methods.

The V_{H} and V_{L} clones obtained were screened for inserts of the expected size by PCR and the DNA sequence of selected clones determined by the dideoxy chain termination method according to standard methods.

The DNA and amino acid sequence for the heavy chain V region is shown in FIG. 1. Six independent clones gave the identical sequence. The locations of the complementarity determining regions (CDRs) were determined with reference to other antibody sequences disclosed in Kabat et al. (1991). E11 V_{H} can be assigned to Mouse Heavy Chains Subgroup IA (Kabat et al., 1991).

The DNA and amino acid sequence for the light chain V region is shown in FIG. 2. Five independent clones gave the identical sequence. The locations of the CDRs were determined with reference to other antibody sequences (Kabat et al., 1991) as disclosed above. E11 V_{L} can be assigned to Mouse Kappa Chains Subgroup III (Kabat et al., 1991).

Two aberrant non-productive light chain sequences, derived from the fusion partner, were also present in the hybridoma.

Design of Immunogenicity-Reduced Antibody Sequences

The murine V_{H} and V_{L} sequences were compared to directories of human germline antibody genes (Cox et al., 1994; Tomlinson et al., 1992). The closest match human germline gene selected as reference for the immunogenicity-reduced V_{H} was DP-65 with J_{H}6. The closest match human germline gene selected as reference for the immunogenicity-reduced V_{L} was b1 with J_{L}5. The murine V region sequences obtained were subjected to peptide threading to identify potential T-cell epitopes, through analysis of binding to 18 different human MHC class II allotypes. The sequences were also analyzed for presence of known human T-cell binding peptides from a database (The Walter and Eliza Hall Institute of Medical Research, Melbourne, Australia, World Wide Web site wehil.wehi.edu.au) using the proprietary computer program “Searcher.”

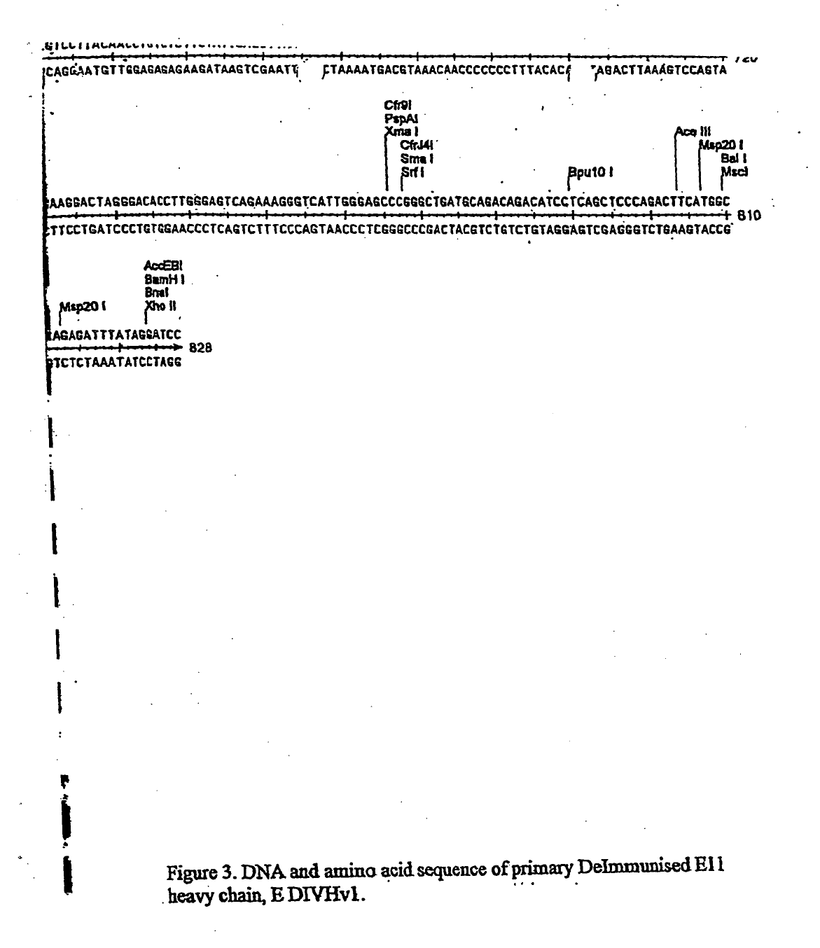

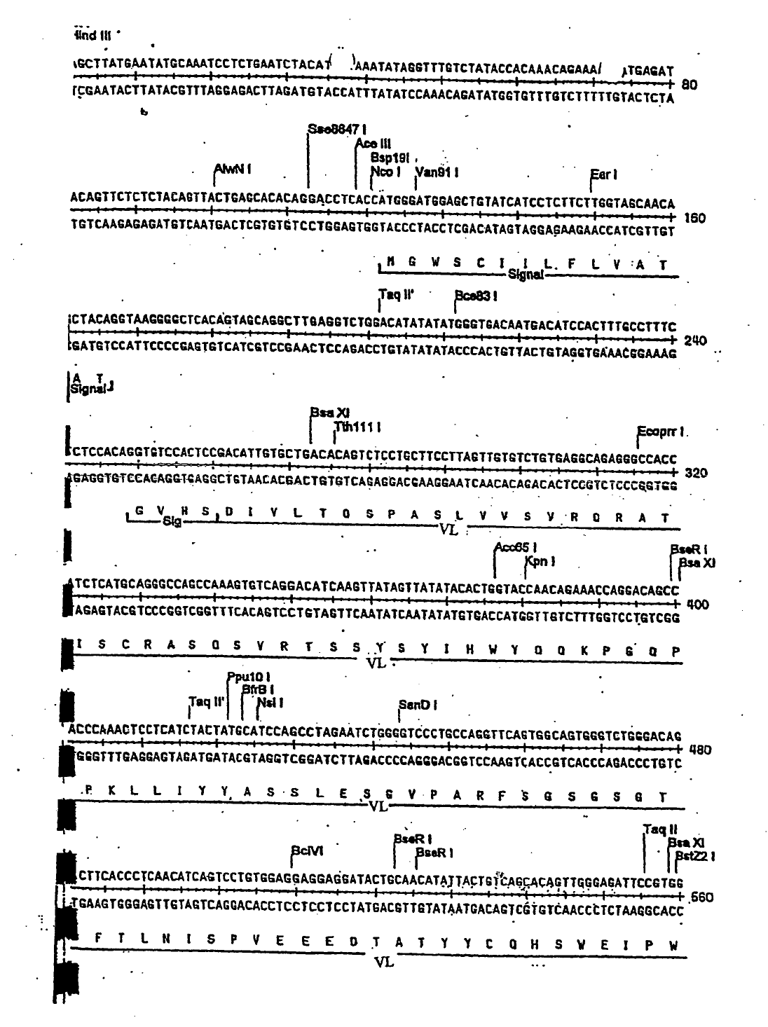

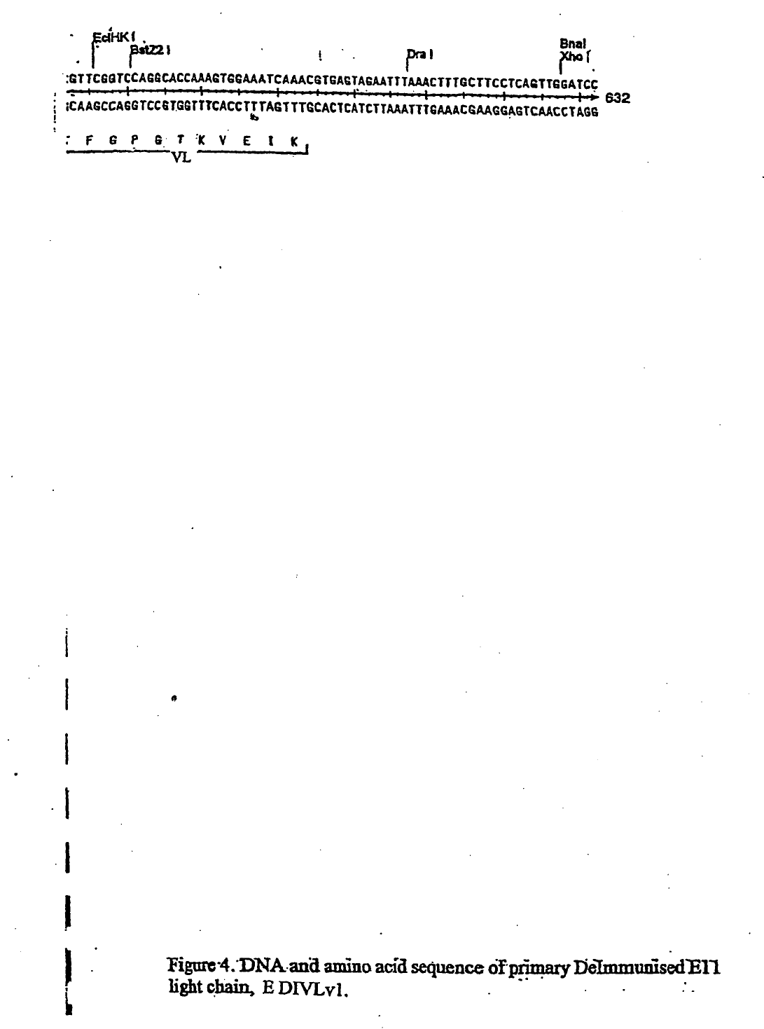

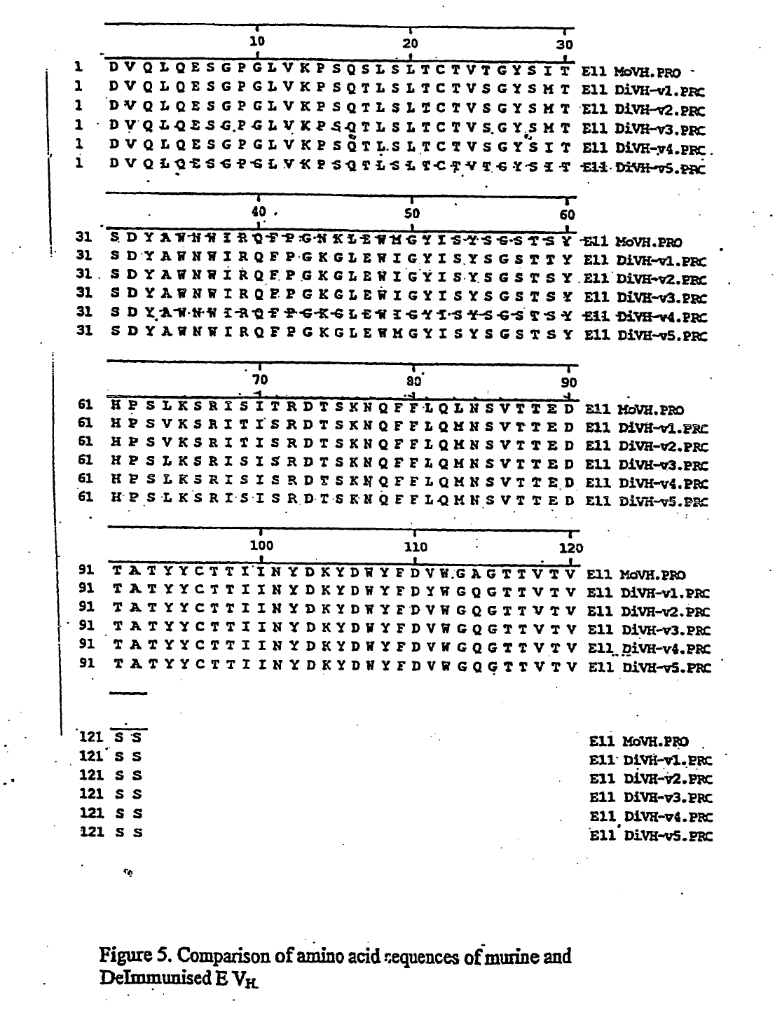

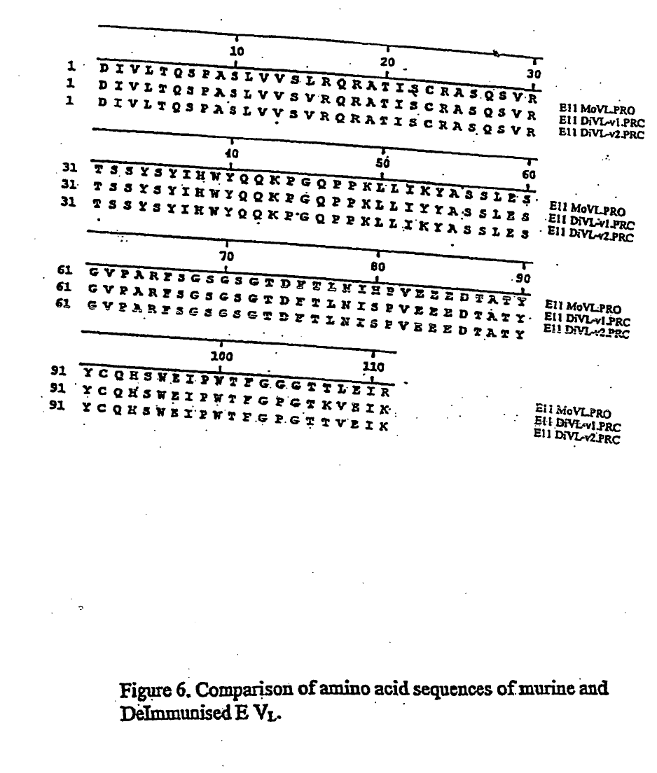

Primary immunogenicity-reduced V_{H} and V_{L} sequences were designed to retain various preferred murine amino acids (EDIVHv1, EDIVLv1). As generation of the primary immunogenicity-reduced sequences requires a small number of amino acid substitutions that might affect the binding of the final immunogenicity-reduced molecule, four other variant V_{H} sequences and one other V_{L} were designed. The DNA sequence for the primary immunogenicity-reduced V_{H} region is shown in FIG. 3 and for the primary immunogenicity-reduced V_{L} in FIG. 4. The comparative amino acid sequences of the murine and immunogenicity-reduced V regions are shown in FIG. 5 for V_{H} and FIG. 6 for V_{L}.

Construction of Immunogenicity-Reduced Antibody Sequences

The immunogenicity-reduced variable regions were constructed by the method of overlapping PCR recombination. The cloned murine V_{H} and V_{L} genes were used as templates for mutagenesis of the framework regions to the required immunogenicity-reduced sequences. Sets of mutagenic primer pairs were synthesized encompassing the regions to be altered. The vectors VH-PCR1 and VL-PCR1 (Riechmann et al., 1988) were used as templates to introduce a 5′ flanking sequence, including the leader signal peptide, leader intron and the murine immunoglobulin promoter, and a 3′ flanking sequence, including the splice site and intron sequences. The immunogenicity-reduced V regions produced were cloned into pUC19 and the entire DNA sequence was confirmed to be correct for each immunogenicity-reduced V_{H} and V_{L}.

Using the above-described methods, the following plasmid DNAs encoding immunogenicity-reduced antibody V regions were created:

pUC19 E DIVH1

pUC19 E DIVH2

pUC19 E DIVH3

pUC19 E DIVH4

pUC19 E DIVH5

pUC19 E DIVL1

pUC19 E DIVL2

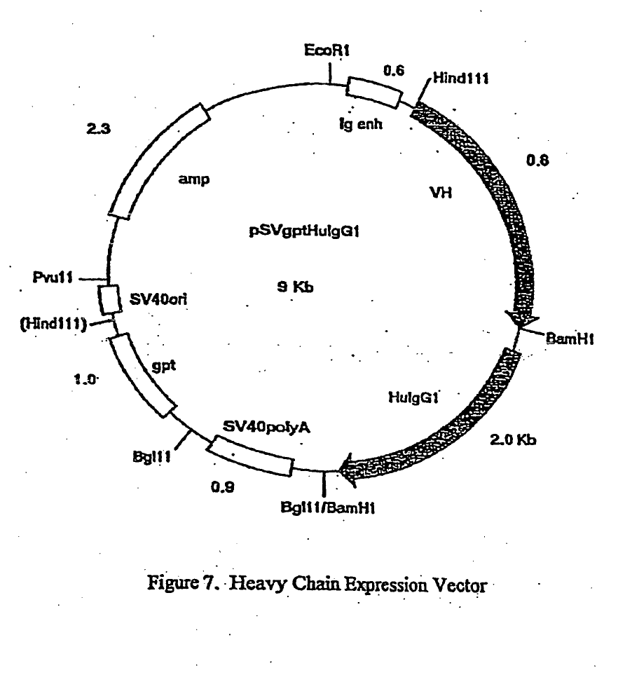

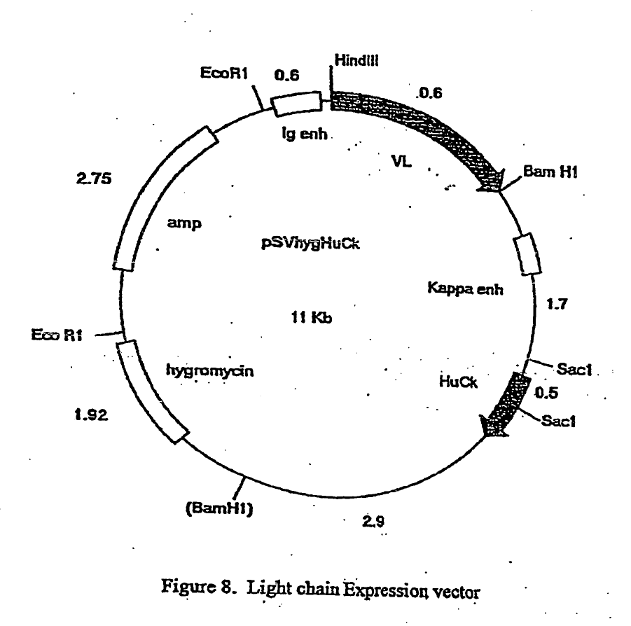

The immunogenicity-reduced heavy and light chain V-region genes were excised from pUC19 as HindIII to BamHI fragments, which include the murine heavy chain immunoglobulin promoter, the leader signal peptide, leader intron, the V_{H} or V_{L} sequence and the splice site. These were transferred to the expression vectors pSVgpt and pSVhyg (FIGS. 7 and 8), which include human IgG1 or 6 constant regions, respectively, and markers for selection in mammalian cells. The DNA sequence was confirmed to be correct for the immunogenicity-reduced V_{H} and V_{L} in the expression vectors.

Construction of Chimeric Antibody Genes

A chimeric antibody consists of human constant regions linked to murine variable regions. A chimeric antibody provides a very useful tool for (1) confirmation that the correct variable regions have been cloned, (2) use as a control antibody in antigen binding assays with the same effector functions and utilizing the same secondary detection reagents as the immunogenicity-reduced (humanized) antibody. Chimeric heavy and light chain expression vectors have been constructed consisting of the E11 murine variable regions linked to human IgG1 or 6 constant regions in the expression vectors pSVgpt and pSVhyg as described by Orlandi et al. (1989). The vectors VH-PCR1 and VL-PCR1 (Riechmann et al., 1988) were used as templates to introduce 5′ flanking sequence including the leader signal peptide, leader intron and the murine immunoglobulin promoter, and 3′ flanking sequence including the splice site and intron sequences. The DNA sequences were confirmed to be correct for the V_{H} and V_{L} in the chimeric expression vectors.

Expression of Immunogenicity-Reduced and Chimeric Antibodies

The host cell line for antibody expression was NS0, a non-immunoglobulin producing mouse myeloma, obtained from the European Collection of Animal Cell Cultures, Porton UK (ECACC No 85110505). The heavy and light chain expression vectors were co-transfected in a variety of combinations into NS0 cells by electroporation. Colonies expressing the gpt gene were selected in Dulbecco's Modified Eagle's Medium (DMEM) supplemented with 10% fetal bovine serum, 0.8 μg/ml mycophenolic acid and 250 μg/ml xanthine. Production of human antibody by transfected cell clones was measured by ELISA for human IgG. Cell lines secreting antibody were selected and expanded. Immunogenicity-reduced and chimeric antibodies were purified using Prosep®-A (Bioprocessing Ltd).

Using the above-described methods, the following cell lines that express immunogenicity-reduced antibodies were produced:

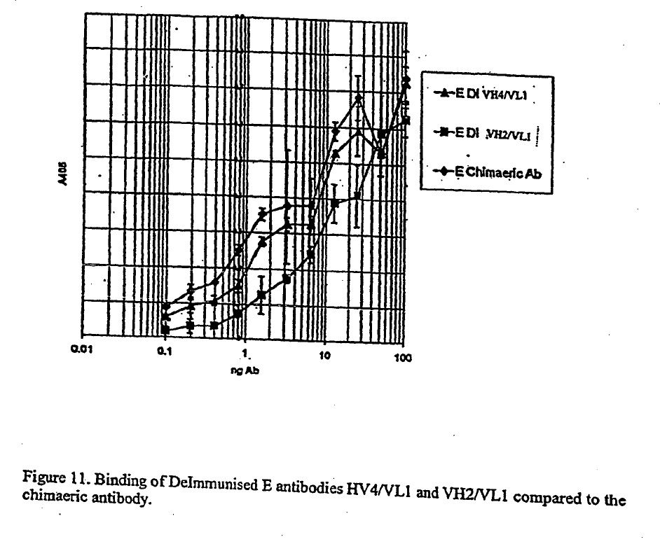

E DI VH4/VL1 19E9, which produces immunogenicity-reduced Ab VH4/VL1 (“E DI VH4VL1”; see FIG. 11).

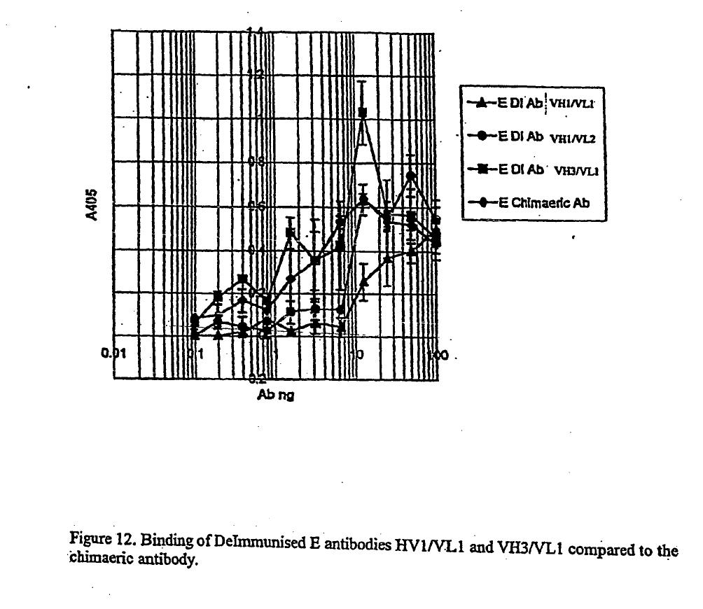

E DI VH3/VL1 12H10, which produces immunogenicity-reduced Ab VH3/VL1 (“E DI VH3/VL1”; see FIG. 12).

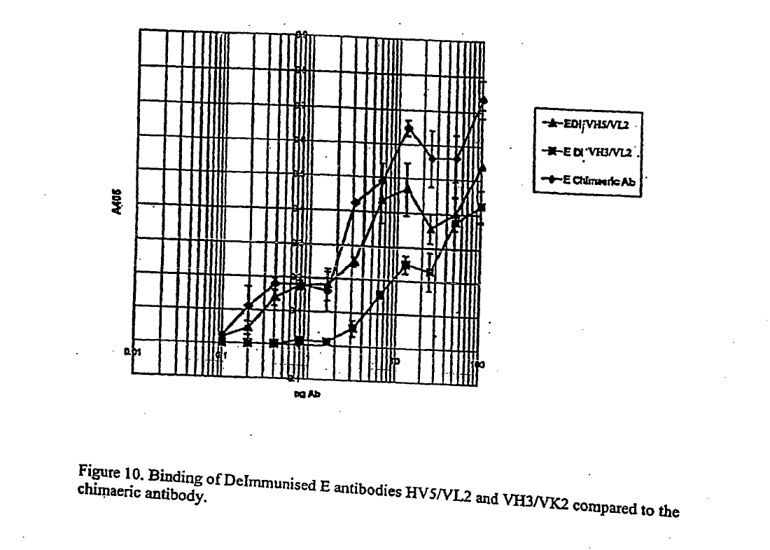

E DI VH3/VL2 15A12, which produces immunogenicity-reduced Ab VH3/VL2 (“E DI VH3/VL2”; see FIG. 10).

E DI VH2/VL1 44H1, which produces immunogenicity-reduced Ab VH2/VL1 (“E DI VH2/VL1”;see FIG. 11).

E DI VH5/VL2 31C11, which produces immunogenicity-reduced Ab VH5/VL2 (“E DI VH5/VL2”; see FIG. 10).

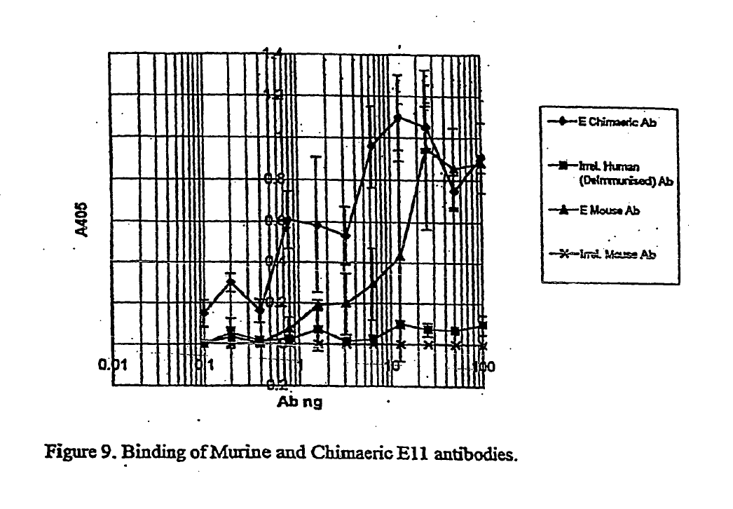

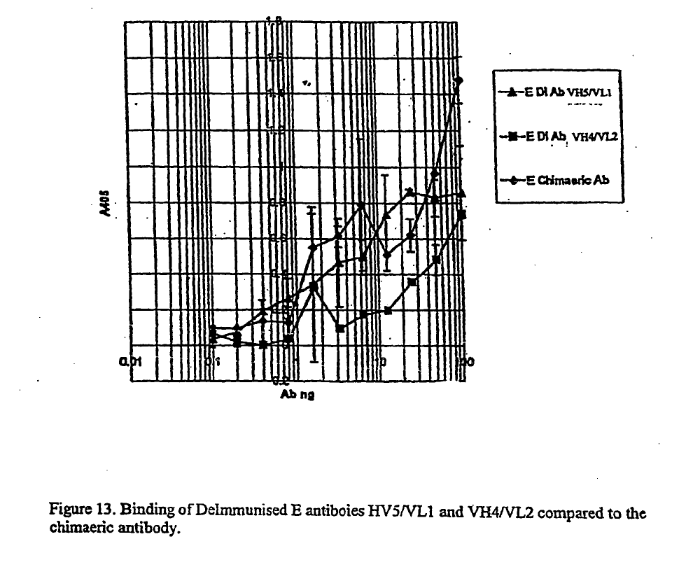

E Ch VH/ChVLA 3G4 (chimeric), which produces immunogenicity-reduced chimeric Ab VH5/VL2(“E Chimaeric Ab”; see FIGS. 9-13).

Antigen Binding Assay

In a pilot antigen binding assay, erythrocytes were fixed to 96-well plates with poly L-lysine and glutaraldehyde. The drawback of fixing erythrocytes to 96-well plates was that it yielded a very high background, possibly caused by denaturation or masking of the antigen on the erythrocytes.

A modified antigen binding assay was therefore adopted wherein the antibodies were reacted with RBCs in solution and the cells only fixed at the end of the assay, just prior to the addition of the substrate. Washed erythrocytes were added to dilutions of antibody (in duplicate or triplicate) in 96-well V-bottom plates. Bound antibody was detected with biotinylated anti-human or anti-mouse antibody, then visualized using avidin alkaline phosphatase according to standard methods. After fixing with glutaraldehyde, color was developed with PNPP substrate and the absorbance read at 405 nm. FIG. 9 shows binding of the murine and chimeric antibodies compared to an irrelevant murine antibody control and an irrelevant human (immunogenicity-reduced) antibody control. Note that the secondary biotinylated reagent is different for the murine and the human (chimeric and immunogenicity-reduced) antibodies such that a direct comparison was not possible.

The results show that both murine and chimeric E11 antibodies bind well and that there is no binding by the irrelevant control antibodies. The chimeric antibody with murine V regions linked to human constant regions was expected to be equivalent to the murine antibody in binding and provided a control for the binding experiments with the immunogenicity-reduced antibodies.

FIGS. 10, 11, 12 and 13 show binding of the immunogenicity-reduced antibodies compared to the chimeric antibody (“E Chimaeric Ab”). immunogenicity-reduced (“DI”) antibodies E DI VH5/VL2, E DI VH5/VL1, E DI VH4/VL1, E DI VH5/VL2 and E DI VH3/VL1 showed equivalent binding to the chimeric antibody. Binding by E DI VH2/VL1 was reduced by approximately two-fold compared to the chimeric antibody. Binding by E DI VH1VL1, E DI VH1/VL2, E DI VH3/VL2 and E DI V4/VL2 was further reduced to approximately ten-fold compared to the chimeric antibody. Tabulated results are shown in Table 1 below. Results are given in ng of antibody at A⁴⁰⁵ 0.4.

TABLE 1
Binding of immunogenicity-reduced
Antibodies to CR1 on erythrocytes.

| | VH5 | VH4 | VH3 | VH2 | VH1 | Chimeric | Mouse |
|---|---|---|---|---|---|---|---|
| | (ng) | (ng) | (ng) | (ng) | (ng) | (ng) | (ng) |
| VL1 | 2 | 4, 7, 3 | 4 | 12 | 50 | 6, 6, 3, 1.2, 4, | 10, 4 |
| VL2 | 3, 5, 3 | 30 | 20, 5 | NA | 9 | 1, 4 | |

These results indicate that immunogenicity-reduced anti-CR1 monoclonal antibodies E DI VH5/VL2, E DI VH5/VL1, E DI VH4/VL1, E DI VH5/VL2 and E DI VH3/VL1 may be used to create heteropolymers (HP) of an immunogenicity-reduced anti-CR1 monoclonal antibody x anti-pathogen monoclonal antibody. Such bispecific antibodies can be used for removing pathogenic agent from the circulation of a human.

### References

Cox J P L, Tomlinson I M, Winter G. A directory of human V_{K} segments reveals a strong bias in their usage. Eur. J. Immunol. 1994; 24: 827-36.

Kabat E A, Wu T T, Perry H M, Gottesman K S, Foeller C.; Sequences of proteins of Immunological Interest, US Department of Health and Human Services, 1991.

Orlandi R, Gussow D, Jones P, Winter G. Cloning immunoglobulin variable domains for expression by the polymerase chain reaction. Proc Natl Acad Sci USA 1989; 86: 3833-7.

Riechmann L, Clark M, Waldmann H, Winter G. Reshaping human antibodies for therapy. Nature 1988; 332: 323-7.

Tomlinson I M, Walter G, Marks J D, Llewelyn M B, Winter G. The repertoire of human germline V_{H} sequences reveals about fifty groups of V_{H} segments with different hypervariable loops. J. Mol. Biol. 1992:227:776-798.

### 6.2. Example 2

### Bispecific Molecule 3F3/19E9

This example illustrates the effects of a monoclonal antibody 3F3 which binds the protective antigen of anthrax and a bispecific molecule comprising 3F3/19E9 on J774 macrophage.

Materials and Reagents

Monkey Erythrocytes: baboon blood in Naeoia from Lampine Bio Labs, Cat # B1-180N-10, Lot # 102938800 (#4). Macrophage cells: J774A1, passage #3, viability was 94.8%, passed at 2×10⁶ cells/ml. rPA (2.2 mg/ml), Lot # 102-72 (aliquoted by CF) NB199-20, diluted 1:100 (2 μl aliquot+198 μl DMEM). Lethal factor (LF) (1.45 mg/ml), Lot # 199-38. It was diluted 1:100 (2 μl aliquot+198 μl DMEM). Shaking speed was 2.1. HP sample: H4-19E9×3F3 MAb (PEG), Lot # 175-91A, concentration was 309.4 μg/ml. The bispecific molecule was produce by cross-linking an immunogenicity-reduced anti-CR1 MAb, 19E9, and a non-neutralizing anti-PA antibody, 3F3, using N-succinimidyl S-acetyl thioacetate (SATA) and NHS-poly (ethylene glycol)-maleimide (PEG-MAL) as the cross-linking agents.

Procedure

1. Diluted HP as below (based on molar ratio of PA): add 50 μl to set with erythrocytes (100%). To the two sets without erythrocytes, add only 25 μl of the MAb as described in Table 2 below and then add 25 μl of DMEM (50%).

TABLE 2

| | Final | Working stock | | |
|---|---|---|---|---|
| | Concentration | concentration | | |
| HP 3F3 | (ng/ml) | (μg/ml) | μl of HP | dDMEM |
| 3x | 1627 | 13.02 | 42.1 | 857.9 |
| 2x | 1664 | 8.67 | 646.7 | 333.36 |
| 1x | 542.2 | 4.34 | 400 of 2x | 400 |
| 0.5x | 271.1 | 2.17 | 400 of 1x | 400 |
| 0.25x | 135.5 | 1.06 | 400 of 0.5x | 400 |
| 0.125x | 67.8 | 0.54 | 400 of 0.25x | 400 |

2. dilution of lethal toxin and HP protection in tubes (FACS);
3. PA washing: the final concentration of rPA (2.2 mg/ml) in cells was 150.0 ng/ml, stock of PA was 0.022 mg/ml (1:100 dilution). The washing was 8×150 ng/ml-1.2 μg/ml, added 163.6 ÿl of PA stock (22 ÿl/ml) to 3 ml of cDMEM;
4. LF washing: the final concentration of LF (1.45 mg/ml) in cells was 150.0 ng/ml, the stock of LF was 14.5 ÿg/ml, the washing was 8×150 ng/ml-1.2 μg/ml, add 245.3 ÿl of LF stock (14.5 ÿg/ml) to 3 ml cDMEM;
5. incubated set with erythrocytes with HP for 45 min. in 37° C. incubator. After incubation, washed 1½time with PBS/BSA;
6. meanwhile, prepared the other 2 sets. After 1½wash for set with erythrocytes, added PA+LF to all tubes at the same time;
7. incubated for 1 hr in 37° C. incubator at a shaking speed of 2.1;
8. added 200 μl of cells and incubated at 37° C. for 3.5 hrs at a shaking speed of 2.1.
9. after a 3.5 hr incubation, took cells out from the shaker. Washed ½times with cold PBS/0.5% BSA buffer;
10. added 200 μl of BD FACS lysing solution to all the tubes and incubated at room temperature for 10 min;
11. incubated at 4° C. for 20 min. and washed 1½ times;
12. added 2 ml of BD FACS lysing solution to all the tubes and incubates at room temperature for 10 min.;
13. washed 1½ times with cold buffer and incubated the final pellet in 400 μl of buffer;
14. analyzed on the FACS calibur within 1 hour.
Results

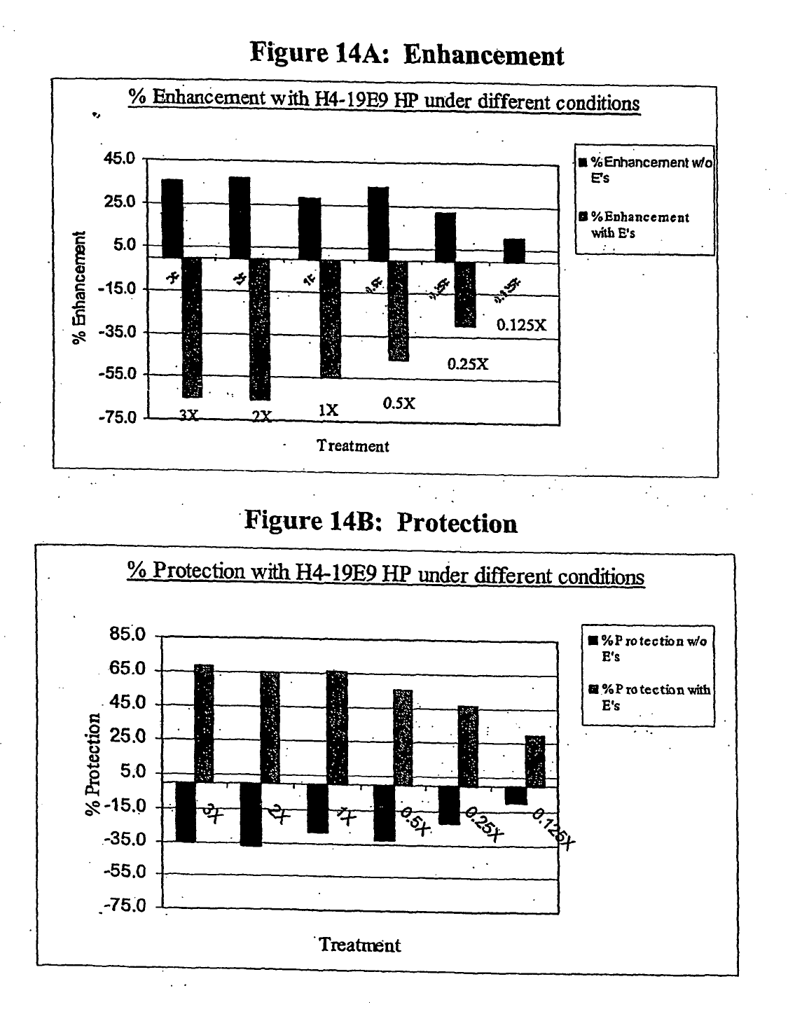

The percentage of enhancement and the percentage of protection of the bispecific molecule 19E9 cross-linked to 3F3 under different conditions are shown in Table 3 and FIGS. 14A and 14B.

TABLE 3

| | | | | | | | Mean w/. | | | | | |
|---|---|---|---|---|---|---|---|---|---|---|---|---|
| | | | | | | | Background | | | | | |
| | Set 1 | | Set 2 | | Mean | | subt. | | % Enhancement | | % protection | |
| | w/o E's | with E's | w/o E's | with E's | w/o E's | with E's | w/o E's | with E's | w/o E's | with E's | w/o E's | with E's |
| Cells only | 0.58 | 0.26 | 0.37 | 1.29 | 0.48 | 0.78 | 0.0 | 0.0 | | | | |
| LeTx | 69.20 | 44.90 | 70.90 | 51.90 | 70.05 | 48.40 | 69.6 | 47.6 | 0.0 | 0.0 | 0.0 | 0.0 |
| 3X | 93.40 | 16.60 | 95.80 | 15.10 | 94.60 | 15.85 | 94.1 | 15.1 | 35.2 | −68.3 | −35.2 | 68.3 |
| 2X | 96.60 | 17.90 | 94.90 | 16.90 | 95.75 | 17.40 | 95.3 | 16.6 | 36.9 | −65.1 | −36.9 | 65.1 |
| 1X | 87.90 | 19.30 | 91.50 | 14.80 | 89.70 | 17.05 | 89.2 | 16.3 | 28.2 | −65.8 | −28.2 | 65.8 |
| 0.5X | | 21.60 | 93.20 | 23.10 | 93.20 | 22.35 | 92.7 | 21.6 | 33.2 | −54.7 | −33.2 | 54.7 |
| 0.25X | | 25.2 | 85.6 | 27.7 | 85.6 | 26.45 | 85.1 | 25.7 | 22.3 | −46.1 | −22.3 | 46.1 |
| 0.125X | | 37.00 | 77.30 | 31.60 | 77.30 | 34.30 | 76.8 | 33.5 | 10.4 | −29.6 | −10.4 | 29.6 |

### CONCLUSION

The data clearly shows that bispecific molecule 3F3/19E9 (HP) protects macrophages from the lethal toxin.

### 7. REFERENCES CITED

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1. A molecule that specifically binds CR1, said molecule comprising amino acids sequence as described by SEQ ID NO: 2, but with one or more of the following amino acid substitutions in SEQ ID NO: 2:
Position 17: Ser→Thr;
Position 25: Thr→Ser;
Position 29: Ile→Met;
Position 44: Asn→Lys;
Position 45: Lys→Gly;
Position 49: Met→Ile;
Position 59: Ser→Thr;
Position 64: Leu→Val;
Position 69: Ser→Thr;
Position 71: Thr→Ser;
Position 83: Leu→Met;
Position 111: Val→Tyr; and
Position 114: Ala→Gln.

2. The molecule of claim 1 that has the following amino acid substitutions in SEQ ID NO: 2:
Position 17: Ser→Thr;
Position 25: Thr→Ser;
Position 29: Ile→Met;
Position 44: Asn→Lys;
Position 45: Lys→Gly;
Position 49: Met→Ile;
Position 59: Ser→Thr;
Position 64: Leu→Val;
Position 69: Ser→Thr;
Position 71: Thr→Ser;
Position 83: Leu→Met;
Position 111: Val→Tyr; and
Position 114: Ala→Gln.

3-6. (canceled)

7. A molecule that specifically binds CR1, said molecule comprising an immunoglobulin variable region comprising a complementarity determining region 2 having an amino acid sequences as described by amino acid numbers 51-66 in SEQ ID NO: 2 but with one or more of the following amino acid substitutions:
Position 59: Ser→Thr; and
Position 64: Leu→Val.

8. A molecule that specifically binds CR1, said molecule comprising an immunoglobulin variable region comprising a complementarity determining region 3 having an amino acid sequences as described by amino acid numbers 99-112 of SEQ ID NO: 2, but with the following amino acid substitution in SEQ ID NO: 2:
Position 111: Val→Tyr.

9. A molecule that specifically binds CR1, said molecule comprising an immunoglobulin variable region comprising:
(a) a complementarity determining region 1 as described by amino acid numbers 31-36 of SEQ ID NO: 2;
(b) a complementarity determining region 2 as described by amino acid numbers 51-66 of SEQ ID NO: 2, but with one or more of the following amino acid substitutions:
Position 59: Ser→Thr, and
Position 64: Leu→Val; and
(c) a complementarity determining region 3 as described by amino acid numbers 99-112 of SEQ ID NO: 2, but with the following amino acid substitution:
Position 111: Val→Tyr.

10. The molecule of claim 1, further comprising amino acids sequence as described by SEQ ID NO: 4, but with one or more of the following amino acid substitutions:
Position 15: Leu→Val;
Position 53: Lys→Tyr;
Position 80: His→Ser;
Position 104: Gly→Pro;
Position 107: Thr→Lys;
Position 108: Leu→Val; and
Position 111: Arg→Lys.

11. The molecule of claim 1, further comprising amino acids sequence as described by SEQ ID NO: 4, but with:
Position 15: Leu→Val;
Position 53: Lys→Tyr;
Position 80: His→Ser;
Position 104: Gly→Pro;
Position 107: Thr→Lys;
Position 108: Leu→Val; and
Position 111: Arg→Lys.

12. (canceled)

13. The molecule of claim 1 that is an immunoglobulin.

14. The molecule of claim 1 that is an scFv.

15. The molecule of claim 1 that is humanized.

16. The molecule of claim 1 that is chimeric.

17. The molecule of claim 1 that is a purified immunoglobulin.

18. A hybridoma expressing the molecule of claim 1, wherein the molecule is an immunoglobulin.

19. A molecule comprising:
(a) a first binding portion that specifically binds pathogenic antigenic molecule desired to be reduced in amount in the circulatory system of a mammal; and
(b) a second binding portion that specifically binds CR1, said second binding portion comprising an amino acid sequence as described by SEQ ID NO: 2, but with one or more of the following amino acid substitutions in SEQ ID NO: 2:
Position 17: Ser→Thr;
Position 25: Thr→Ser;
Position 29: Ile→Met;
Position 44: Asn→Lys;
Position 45: Lys→Gly;
Position 49: Met→Ile;
Position 59: Ser→Thr;
Position 64: Leu→Val;
Position 69: Ser→Thr;
Position 71: Thr→Ser;
Position 83: Leu→Met;
Position 111: Val→Tyr; and
Position 114: Ala→Gln.

20. The molecule of claim 19 that has the following amino acid substitutions in SEQ ID NO: 2:
Position 17: Ser→Thr;
Position 25: Thr→Ser;
Position 29: Ile→Met;
Position 44: Asn→Lys;
Position 45: Lys→Gly;
Position 49: Met→Ile;
Position 59: Ser→Thr;
Position 64: Leu→Val;
Position 69: Ser→Thr;
Position 71: Thr→Ser;
Position 83: Leu→Met;
Position 111: Val→Tyr; and
Position 114: Ala→Gln.

21-24. (canceled)

25. The molecule of claim 19, wherein said second binding portion further comprises amino acid sequence as described by SEQ ID NO: 4, but with one or more of the following amino acid substitutions in SEQ ID NO: 4:
Position 15: Leu→Val;
Position 53: Lys→Tyr;
Position 80: His→Ser;
Position 104: Gly→Pro;
Position 107: Thr→Lys;
Position 108: Leu→Val; and
Position 111: Arg→Lys.

26. The molecule of claim 19, wherein said second binding portion further comprises amino acid sequence as described by SEQ ID NO: 4, but that has the following amino acid substitutions in SEQ ID NO: 4:
Position 15: Leu→Val;
Position 53: Lys→Tyr;
Position 80: His→Ser;
Position 104: Gly→Pro;
Position 107: Thr→Lys;
Position 108: Leu→Val; and
Position 111: Arg→Lys.

27. (canceled)

28. The molecule of claim 19, wherein said second binding portion is an immunoglobulin or an Fab region thereof.

29. The molecule of claim 19, wherein said second binding portion is an immunoglobulin or an Fab region thereof and said first binding portion is an immunoglobulin or an Fab region thereof.

30. The molecule of claim 19, wherein said second binding portion is an immunoglobulin or an Fab region thereof, said first binding portion is an immunoglobulin or an Fab region thereof, and said first and second binding portions are cross-linked to each other.

31-33. (canceled)

34. The molecule of claim 19, wherein said second binding portion is an immunoglobulin or an Fab region thereof.

35. The molecule of claim 19, wherein said second binding portion is an immunoglobulin or an Fab region thereof and said first portion is an immunoglobulin or an Fab region thereof.

36. The molecule of claim 19, wherein said first and second binding portions are cross-linked to each other.

37-39. (canceled)

40. A molecule comprising:
(a) a first binding portion that specifically binds (i) an antigen of a pathogen;
(ii) an autoantigen; or
(ii) a blood-borne protein desired to be removed from the circulatory system of a mammal; and
(b) a second binding portion that specifically binds CR1, said binding portion comprising an immunoglobulin variable region comprising a complementarity determining region 2 as described by amino acid numbers 51-66 of SEQ ID NO: 2, but with one or more of the following amino acid substitutions in SEQ ID NO: 2:
Position 59: Ser→Thr; and
Position 64: Leu→Val.

41. The molecule of claim 40 that has the following amino acid substitutions in SEQ ID NO: 2:
Position 59: Ser→Thr; and
Position 64: Leu→Val.

42. The molecule of claim 40, said immunoglobulin variable region comprising a complementarity determining region 1 as described amino acid numbers 31-36 of SEQ ID NO: 2.

43. A molecule comprising:
(a) a first binding portion that specifically binds
(i) an antigen of a pathogen;
(ii) an autoantigen; or
(ii) a blood-borne protein desired to be removed from the circulatory system of a mammal; and
(b) a second binding portion that specifically binds CR1, said binding portion an immunoglobulin variable region comprising a complementarity determining region 3 as described by amino acid numbers 99-112 of SEQ ID NO: 2, but with the following amino acid substitution in SEQ ID NO: 2:
Position 111: Val→Tyr.

44. The molecule of claim 43, said immunoglobulin variable region comprising a complementarity determining region 1 as described by amino acid numbers 31-36 of SEQ ID NO: 2.

45-49. (canceled)

50. The molecule of claim 19 that is a dimeric molecule comprising a first polypeptide and a second polypeptide, wherein the first polypeptide comprises the first binding domain and the second polypeptide comprises the second binding domain, and wherein the first polypeptide and the second polypeptide is each independently selected from the group consisting of (a) a third polypeptide consisting essentially of, in amino- to carboxy-terminal order, an immunoglobulin variable light chain domain, an immunoglobulin constant light chain domain, a linker polypeptide, an immunoglobulin variable heavy chain domain, a CH1 domain, an immunoglobulin hinge region, a CH2 domain, and a CH3 domain; and (b) a fourth polypeptide consisting essentially of, in amino- to carboxy-terminal order, a scFv, a CH1 domain, an immunoglobulin hinge region, a CH2 domain, and a CH3 domain.

51. (canceled)

52. The molecule of claim 19 that is a polypeptide, said polypeptide consisting essentially of, in amino- to carboxy-terminal order, a first polypeptide and a second polypeptide, wherein the first polypeptide comprises the first binding domain and the second polypeptide comprises the second binding domain, and wherein the first polypeptide consists essentially of, in amino- to carboxy-terminal order, a first scFv, a CH2 domain, and a CH3 domain; and the second polypeptide consists essentially of, in amino- to carboxy-terminal order, a second scFv domain.

53. (canceled)

54. The molecule of claim 19 that is a polypeptide, said polypeptide consisting essentially of, in amino- to carboxy-terminal order, a first polypeptide and a second polypeptide, wherein the first polypeptide comprises the first binding domain and the second polypeptide comprises the second binding domain, and wherein the first polypeptide consists essentially of, in amino- to carboxy-terminal order, a first scFv, a CH3 domain, and a CH2 domain; and the second polypeptide consists essentially of, in amino- to carboxy-terminal order, a second scFv domain.

55-56. (canceled)

57. A method for removing a blood-borne antigen, autoantigen or pathogen from the circulation of a mammal comprising administering to said mammal an amount of the molecule of claim 19, effective to remove the antigen of interest from the circulation of the mammal.

58. A method for removing a blood-borne antigen, autoantigen or pathogen from the circulation of a human comprising administering to said human an amount of the molecule of claim 19, effective to remove the antigen of interest from the circulation of the human.

59. A method for removing a blood-borne antigen, autoantigen or pathogen from the circulation of a mammal, wherein the antigen, autoantigen or pathogen is expressed in the circulation of said mammal, said method comprising administering to said mammal an amount of the molecule of claim 19, effective to remove the antigen of interest from the circulation of the mammal.

60. A method for removing a blood-borne antigen autoantigen or pathogen from the circulation of a human, wherein the antigen, autoantigen or pathogen is expressed in the circulation of said human, said method comprising administering to said human an amount of the molecule of claim 19, effective to remove the antigen of interest from the circulation of the human.

61. A pharmaceutical composition comprising a therapeutically effective amount of the molecule of claim 19; and a pharmaceutically acceptable carrier.

62. A kit comprising in one or more containers, one or more isolated nucleic acids encoding the molecule of claim 19.

63. A kit comprising in one or more contained a cell transformed with one or more nucleic acids encoding molecule of of claim 19.
